(19) United States
(12) Patent Application Publication
(10) Pub. No.: US 2026/0166178 A1
(43) Pub. Date: Jun. 18, 2026

(54) MANIPULATING GOLGI IONS FOR TREATMENT OF NEURODEGENERATIVE DISEASE AND CUTANEOUS DISORDERS

(71) Applicants: The Johns Hopkins University, Baltimore, MD (US); Formation Venture Engineering Foundry, Inc., Beverly, MA (US)

(72) Inventors: Rajini Rao, Baltimore, MD (US); Ruby Gupta, Baltimore, MD (US); Ajay Verma, Beverly, MA (US)

(21) Appl. No.: 19/543,607

(22) Filed: Feb. 18, 2026

Related U.S. Application Data
(63) Continuation-in-part of application No. PCT/US2025/042442, filed on Aug. 18, 2025.
(60) Provisional application No. 63/684,798, filed on Aug. 19, 2024.

(51) Int. Cl. A61K 48/00; A61K 38/43; A61P 25/28; C12N 15/86

(52) U.S. Cl. CPC A61K 48/005; A61K 38/43; A61P 25/28; C12N 15/86; C12N 2750/14143

(57) ABSTRACT

The invention provides that the expression and/or activity of metal ion transporter, such as a Ca²⁺/Mn²⁺ transporter and secretory pathway calcium ATPase 1 (SPCA1) or SPCA2 is associated with the restoration and conservation of Golgi integrity in cells, as well as with the restoration of Golgi ion concentrations. Provided herein are methods of restoring or maintaining Golgi integrity in a cell and methods of modulating Golgi ion concentration is a cell including increasing the expression and/or activity of a metal ion transporter such as SPCA1 or SPCA2 protein in the cell. The invention further provides methods of treating neurodegenerative disease, such as amyotrophic lateral sclerosis (ALS). The methods of treatment include increasing the expression and/or activity of a metal ion transporter such as SPCA1 or SPCA2 protein in cells to restore or maintain Golgi integrity and Golgi ion concentration in the cell.

### CROSS-REFERENCE TO RELATED APPLICATIONS

This application is a continuation-in-part application of International Application No. PCT/US2025/042442 filed Aug. 18, 2025, now pending; which claims the benefit under 35 USC § 119(e) to U.S. Application Ser. No. 63/684,798 filed Aug. 19, 2024, now expired. The disclosure of each of the prior applications is considered part of and is incorporated by reference in the disclosure of this application.

### INCORPORATION OF SEQUENCE LISTING

The material in the accompanying sequence listing is hereby incorporated by reference into this application. The accompanying sequence listing xml file, named JHU4750-1_ST26.xml, was created on Feb. 11, 2026 and is 32,472 bytes in size.

### BACKGROUND OF THE INVENTION

### Field of the Invention

The present invention relates generally to the Golgi apparatus and its ionic concentration, and more specifically to methods of modulating Golgi ionic concentration, for use for the treatment of neurodegenerative and cutaneous diseases and disorders.

### Background Information

The Golgi apparatus lies at the crossroads of intracellular trafficking pathways through which all proteins destined for transmembrane, synaptic, axonal, or extracellular locations must pass. During Golgi transit, cargo proteins are modified, sorted and subjected to quality control. The ionic composition of the Golgi is critical for these functions. In many neurodegenerative as well as cutaneous disorders, fragmentation of the Golgi apparatus precedes patient symptoms. There remains an unmet need in the art for means to manipulate Golgi ions (e.g., Ca²⁺) as a way to restore Golgi integrity and protect against cell damage. The present invention provides a means for regulating the activity and/or expression of metal ion transporter, such as SPCA, a Golgi calcium and manganese transport protein, to directly manipulate Golgi ions.

### SUMMARY OF THE INVENTION

The present invention is based on the seminal discovery that the modulation of secretory pathway calcium ATPases (SPCA) expression and/or activity modulates metal ion concentration in the Golgi and restores Golgi integrity and function.

In one embodiment, the present invention provides a method of maintaining and/or restoring Golgi integrity in a cell including increasing the expression and/or activity of a metal ion transporter in the cell, thereby maintaining and/or restoring Golgi integrity.

In one aspect, the metal ion transporter is a Ca²⁺/Mn²⁺ transporter. In another aspect, the Ca²⁺/Mn²⁺ transporter is a secretory pathway calcium ATPase (SPCA) protein. In one aspect, the SPCA protein is SPCA1 or SPCA2. In some aspects, increasing SPCA1 or SPCA2 expression and/or activity in the cell comprises contacting the cell with a nucleic acid encoding a SPCA1 or SPCA2 protein. In other aspects, increasing SPCA1 or SPCA2 expression and/or activity in the cell comprises contacting the cell with a vector comprising a nucleic acid encoding a SPCA1 or SPCA2 protein. In many aspects, vector is an adeno-associated virus (AAV) vector, optionally wherein the AAV vector is an AAV9 vector or an AAV2 vector. In various aspects, the AAV vector includes SEQ ID NO: 7, SEQ ID NO:8, SEQ ID NO:1 or SEQ ID NO:2. In some aspects, increasing SPCA1 or SPCA2 expression and/or activity in the cell comprises contacting the cell with a small molecule agonist or activator of SPCA1 or SPCA2. In one aspect, the cell is a neuronal cell or a skin cell. In some aspect, the neuronal cell is a cell from a subject having a neurodegenerative disease. In various aspects, the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD). In other aspects, the skin cell is a cell from a subject having Hailey Hailey disease (HHD). In some aspects, the cell is from a subject having Brody myopathy (BRM), acrokeratosis verruciformis (AKV), Darier disease (DD), Hailey-Hailey disease (IHID), or a congenital disorder of glycosylation, Type Ilk (CDG2K). In other aspects, the cell is from a subject carrying a mutation is a gene selected from the group consisting of a sarcoplasmic/endoplasmic reticulum calcium ATPase (SERCA), a secretory pathway Ca²⁺-transporting ATPase (SPCA) and TMEM165.

In another embodiment, the invention provides, a method of modulating Golgi ion concentration in a cell including increasing the expression and/or activity of a metal ion transporter in the cell, thereby modulating Golgi ion concentration in the cell.

In one aspect, modulating Golgi ion concentration in the cell includes (i) increasing Ca²⁺ and/or Mn²⁺ concentration in the Golgi, (ii) decreasing Ca²⁺ and/or Mn²⁺ in the cytoplasm, (iii) restoring Golgi Ca²⁺ and/or Mn²⁺ level and/or (iv) increasing cytoplasmic Ca²⁺ and/or Mn²⁺ clearance rate in the cell. In one aspect, the metal ion transporter is a Ca²⁺/Mn²⁺ transporter. In another aspect, the Ca²⁺/Mn²⁺ transporter is a secretory pathway calcium ATPase (SPCA) protein. In one aspect, the SPCA protein is SPCA1 or SPCA2.

In a further embodiment, the invention provides a method of treating a neurodegenerative disease in subject including increasing the expression and/or activity of a metal ion transporter in the subject, thereby treating the neurodegenerative disease.

In one aspect, the metal ion transporter is a Ca²⁺/Mn²⁺ transporter. In another aspect, the Ca²⁺/Mn²⁺ transporter is a secretory pathway calcium ATPase (SPCA) protein. In one aspect, the SPCA protein is SPCA1 or SPCA2. In another aspect, the SPCA protein is SPCA1 or SPCA2. In one aspect, increasing the expression and/or activity of a metal ion transporter in the subject includes increasing SPCA1 or SPCA2 expression and/or activity in a cell from the subject. In one aspect, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof. In another aspect, increasing SPCA1 or SPCA2 expression and/or activity in a cell inhibits the progression of the neurological disease and/or inhibits the development of clinical symptoms. In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector comprising a nucleic acid encoding a SPCA1 or SPCA2 protein. In various aspects, the vector is an AAV vector, optionally wherein the AAV vector is an AAV9 vector. In some aspects, the AAV vector includes SEQ ID NO: 7, SEQ ID NO:8, SEQ ID NO:1 or SEQ ID NO:2. In other aspects, increasing SPCA1 or SPCA2 expression and/or activity in the cell from the subject includes contacting the cell with a small molecule agonist or activator of SPCA1 or SPCA2. In another aspect, increasing SPCA1 or SPCA2 expression and/or activity in the subject includes administering to the subject a vector including a nucleic acid encoding a SPCA1 or SPCA2 protein. In some aspects, the vector is an AAV vector, optionally wherein the AAV vector is an AAV9 vector. In one aspect, increasing SPCA1 expression and/or activity in the subject includes administering to the subject a small molecule agonist or activator of SPCA1 or SPCA2. In some aspects, the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD). In other aspects, the disease is characterized by Golgi fragmentation in neuron cells, mitochondrial membrane depolarization in neuron cells, increased ROS production in neuron cells, stress body (RNA-protein) formation in neuron cells, dysregulation of Ca²⁺ homeostasis in neuron cells, and/or Mn²⁺ sensitivity in neuron cells. In other aspects, the neurodegenerative disease is selected from the group consisting of ALS, AD and PD.

In an additional embodiment, the invention provides a method of treating a disease in a subject including increasing the expression and/or activity of a metal ion transporter in the subject, wherein the disease is selected from the group consisting of Brody myopathy (BRM), acrokeratosis verruciformis (AKV), Darier disease (DD), Hailey-Hailey disease (HHD), or a congenital disorder of glycosylation, Type Ilk (CDG2K), thereby treating the disease in the subject.

In one embodiment, the invention provides a method of treating HHD in a subject comprising increasing the expression and/or activity of a SPCA protein in the subject, thereby treating HHD. In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector comprising a nucleic acid encoding a SPCA1 or SPCA2 protein. In various aspects, the vector is an AAV vector, optionally wherein the AAV vector is an AAV2 vector. In some aspects, increasing SPCA1 or SPCA2 expression and/or activity in the cell from the subject includes contacting the cell with a small molecule agonist or activator of SPCA1 or SPCA2. In another aspect, increasing SPCA1 or SPCA2 expression and/or activity in the subject includes administering to the subject a vector including a nucleic acid encoding a SPCA1 or SPCA2 protein. In some aspects, the vector is an AAV vector, optionally wherein the AAV vector is an AAV2 vector.

In a further embodiment, the invention provides a method of inhibiting TDP-43 cytoplasmic mislocalization, inhibiting TDP-43 cytoplasmic aggregation and/or maintaining TDP-43 nuclear localization in a cell including increasing the expression and/or activity of a metal ion transporter in the cell, thereby inhibiting TDP-43 cytoplasmic mislocalization, inhibiting TDP-43 cytoplasmic aggregation and/or maintaining TDP-43 nuclear localization in the cell.

In a further embodiment, the invention provides a method of treating amyotrophic lateral sclerosis (ALS), in a subject including increasing the expression and/or activity of a secretory pathway calcium ATPase (SPCA) protein in the subject, thereby treating ALS.

In one aspect, increasing SPCA1 or SPCA2 expression and/or activity in the cell from the subject includes contacting the cell with a vector including a nucleic acid encoding a SPCA1 or SPCA2 protein. In various aspects, the vector is an adeno-associated virus (AAV) vector. In some aspects, the vector is an AAV9 vector. In other aspects, the AAV vector comprises SEQ ID NO: 7, SEQ ID NO:8, SEQ ID NO:1 or SEQ ID NO:2. In one aspect, treating ALS comprises improving hindlimb weakness and paralysis, inhibiting forelimb issues, inhibiting muscle atrophy, increasing grip, reducing tremors, improving posture, inhibiting weight loss, and/or inhibiting paralysis in the subject.

### BRIEF DESCRIPTION OF THE DRAWINGS

FIGS. 1A-1D illustrate the ability of SPCA1 to modulate ion concentrations by increasing Ca²⁺ in the Golgi (FIGS. 1A-1B) and correspondingly accelerating the decrease of Ca²⁺ in the cytoplasm (FIGS. 1C-1D). FIG. 1A shows representative images of NSC-34 neurons transfected with SOD1^{WT}, SOD^{G93A} mutant and SOD1^{G93A} mutant plus SPCA1 (+SPCA1) loaded with the fluorescent Ca²⁺ sensor Mag Fluo-4 which accumulates in the Golgi. FIG. 1B quantifies the baseline Ca²⁺-dependent Mag Fluo-4 fluorescence in arbitrary units (a.u.) for the three sets of cells. A total of 90-100 cells per set is represented from 3 independent experiments. FIG. 1C shows representative images (left) and individual cell traces (right) of NSC-34 neurons loaded with the fluorescent Ca²⁺ sensor Fluo-8 which accumulates in the cytoplasm. Thapsigargin releases Ca²⁺ from the endoplasmic reticulum stores and is subsequently cleared by SPCA pumps. The rate of clearance is faster in “+SPCA1” cells, as shown by the slope of the traces plotted in FIG. 1D. 182 cells were analyzed, from 3 independent experiments.

FIGS. 2A-2B illustrate that increasing/boosting SPCA1 expression reverses Golgi fragmentation, as evaluated in SOD1^{WT} cells, SOD1^{G93A} cells, and SOD1^{G93A}+SPCA1 cells. FIG. 2A shows immunofluorescent images showing that SOD1 mutations are associated with Golgi fragmentation, which is reversed when SPCA1 expression is increased. FIG. 2B is a bar graph illustrating the quantification of Golgi fragmentation in cells shown in FIG. 2A.

FIGS. 3A-3B illustrate that increasing/boosting SPCA1 expression reverses mitochondrial depolarization, as evaluated in SOD1^{WT} cells, SOD1^{G93A} cells, and SOD1^{G93A}+SPCA1 cells. FIG. 3A shows histograms illustrating mitochondrial membrane potential as measured by flow cytometry using MitoTracker® Red CMXROS biomarker. FIG. 3B shows the overlap of the histogram of FIG. 3A.

FIGS. 4A-4C illustrate that increasing/boosting SPCA1 expression rescues cells from oxidative stress, as evaluated in SOD1^{WT} cells, SOD1^{G93A} cells, and SOD1^{G93A}+SPCA1 cells. FIG. 4A is a density plot showing the evaluation of the number of death cells, viable cells and cells with high ROS levels in SOD1^{G93A} cells. FIG. 4B is a density plot showing the evaluation of the number of death cells, viable cells and cells with high ROS levels in SOD1^{G93A}+SPCA1 cells. FIG. 4C shows the overlap of histograms illustrating the number of cells with high ROS levels in SOD1^{WT} cells, SOD1^{G93A} cells, and SOD1^{G93A}+SPCA1 cells.

FIG. 5 shows filipin stained images illustrating that increasing/boosting SPCA1 expression reverses cholesterol accumulation in the SOD1 mutant cells.

FIG. 6 shows electron microscopy and immunofluorescent images illustrating the correction of mitochondrial fragmentation in SOD1^{G93A} cells by SPCA1 and SPCA2 constructs.

FIGS. 7A-7B illustrate that boosting SPCA1 expression corrects TDP-43 mislocalization. FIG. 7A shows immunofluorescence images used to evaluate TDP-43 mislocalization visually using representative immunofluorescence images. FIG. 7B is a bar graph illustrating quantitative nuclear to cytoplasmic fluorescence ratios in TDP43 WT cells, TDP43 ANLS cells, and TDP43 ANLS+SPCA1 cells.

FIGS. 8A-8B illustrates the evaluation of the new AAV9 vectors developed for transgenic expression of myc-tagged SPCA1 and SPCA2 in neurons. FIG. 8A is a schematic of the AAV9 constructs. FIG. 8B shows immunofluorescence images illustrating the use of developed SPCA1 AAV9 vector to reverse Golgi fragmentation, as evaluated by comparison of immunofluorescence images of the Golgi marker protein Golgin 97 in SOD1^{WT} cells, SOD1^{G93A} cells, and SOD1^{G93A}+SPCA1 AAV9 vector cells.

FIG. 9 illustrates the expression level of SPCA1 in motor cortex of WT and SOD1-G93A mouse model of ALS. Immunofluorescence images of mouse motor cortex stained for SPCA1 protein show reduced expression in SOD1-G93A mouse model of ALS, compared to the wild type (WT) control in 4-month-old male mice.

FIG. 10 illustrates SPCA1 transgene expression in the brain 2 weeks after the unilateral injection of an AAV9-SPCA1 viral vector into the motor cortex of P1 neonate transgenic SOD1-G93A mice. Immunofluorescence images of mouse motor cortex stained for SPCA1 protein show increased expression.

### DETAILED DESCRIPTION OF THE INVENTION

The present invention is based on the seminal discovery that the modulation of secretory pathway calcium ATPases (SPCA) expression and/or activity modulates metal ion concentration in the Golgi and restores Golgi integrity and function.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of maintaining and/or restoring Golgi integrity in a cell including increasing the expression and/or activity of a metal ion transporter in the cell, thereby maintaining and/or restoring Golgi integrity.

The Golgi apparatus, also known as the Golgi complex, Golgi body, or simply the Golgi, is an organelle found in most eukaryotic cells. Part of the endomembrane system in the cytoplasm, it packages proteins into membrane-bound vesicles inside the cell before the vesicles are sent to their destination. It resides at the intersection of the secretory, lysosomal, and endocytic pathways. It is of particular importance in processing proteins for secretion, containing a set of glycosylation enzymes that attach various sugar monomers to proteins as the proteins move through the apparatus.

The Golgi apparatus is a major collection and dispatch station of protein products received from the endoplasmic reticulum (ER). Proteins synthesized in the ER are packaged into vesicles, which then fuse with the Golgi apparatus. These cargo proteins are modified and destined for secretion via exocytosis or for use in the cell. The Golgi apparatus is also involved in lipid transport and lysosome formation. The structure and function of the Golgi apparatus are intimately linked. Individual stacks have different assortments of enzymes, allowing for progressive processing of cargo proteins as they travel from the cisternae to the trans Golgi face.

By “maintaining” or “restoring” Golgi integrity, it is meant that the methods described herein are used in cases where Golgi integrity has been affected. For example, certain mutations or diseases are associated with Golgi fragmentation, dysregulation of Ca²⁺ homeostasis and Mn²⁺ sensitivity. By “Golgi integrity” it is meant both structural and functional integrity of the Golgi apparatus. In cases where the Golgi integrity may be altered in the future or is suspected to be altered in the future (in cases of genetic predisposition for example), the methods described herein can be used to maintain it (e.g., preventing it from being impaired). In cases where Golgi integrity has been altered, the methods described herein can be used to regain part or all of the Golgi integrity. “restoring” as used herein is meant to include any level of change between an initially altered level and a normal/unaltered level, all the way up to a complete restoration.

Metal ions in the Golgi lumen, such as Ca²⁺, Mn²⁺, Zn²⁺, and Cu²⁺, are important cofactors of many Golgi-residing enzymes. Being an indispensable part of Golgi homeostasis, the maintenance of metal ion homeostasis involves the cooperation of ATPase pumps, channels, and metal ion binding proteins. Mutations in these regulators impair metal ion homeostasis, disrupt cellular activities, and cause human diseases. Regulators/transporters involved in Golgi Ca²⁺, Mn²⁺, Zn²⁺, and Cu²⁺ ion homeostasis have functions under physiological and pathological conditions.

The early Golgi compartments host many Ca²⁺ pumps that are also highly abundant in the ER, such as Sarcoplasmic/endoplasmic reticulum calcium ATPases (SERCAs). The Golgi also contains unique Ca²⁺ transporters not found in the ER, including Secretory pathway Ca²⁺-transporting ATPases (SPCAs, also known as the Calcium-transporting ATPase type 2C members) and TMEM165. Different from ER Ca²⁺ pumps, they usually show high affinity to Mn²⁺ ion and therefore act as Mn²⁺ transporters as well.

In one aspect, the metal ion transporter is a Ca²⁺/Mn²⁺ transporter. In another aspect, the Ca²⁺/Mn²⁺ transporter is a secretory pathway calcium ATPase (SPCA) protein.

Secretory pathway calcium ATPase (SPCA) proteins or secretory pathway Ca²⁺-ATPases are a calcium ATPase-type P-ATPase encoded for by the genes ATP2C1 and ATP2C2. SPCA are found primarily in the membranes of the Golgi apparatus in increasing concentrations from the cis- to the trans-Golgi compartments. Following a calcium spike, SPCA proteins are responsible for transporting Ca²⁺ ions from the cytosol to the lumen of the Golgi, thus lowering the cytoplasmic concentrations of Ca²⁺ to resting levels. SPCA are also able to transport Mn²⁺ ions into the Golgi with high affinity, an ability that the related Ca²⁺-ATPase, SERCA, does not possess. Since Mn²⁺ ions are not used for signaling like Ca²⁺ ions are, the main reason for transporting them out of the cytosol is to prevent manganese toxicity. The removal of these ions from the cytosol can also be looked upon as supplying the Golgi apparatus and thus the entire secretory pathway with these ions. Several proteins within the pathway require either Ca²⁺ ions, Mn²⁺ ions, or divalent ions to function as metal cofactors, such as aminopeptidase P, Proprotein convertases and sulfotransferases.

The SPCA proteins directly transport calcium and manganese ions into the Golgi compartments where they are critical to maintain Golgi integrity and function, including protein processing, glycosylation, sorting and quality control.

The human genome has three SERCA genes, ATP2A1, ATP2A2, and ATP2A3, which encode P2A ATPase family members SERCA1 (SERCA 1a, 1b), 2 (SERCA 2a, 2b), and 3 (SERCA 3 a-e), respectively. The transport of Ca²⁺ from the cytosol into the Golgi lumen by these P-type ATPases is facilitated by the ATP-hydrolysis-dependent conformational change between the two major statuses (E1 and E2) of the SERCA enzymes. The E1 status presents high Ca²⁺-binding affinity and exposure of the two Ca²⁺-binding sites to the cytosol. The binding of Ca²⁺ and ATP causes phosphorylation on a highly conserved aspartate, leading to a conformational change to the E2 status, which shows lower affinity to the Ca²⁺ and reorients toward the lumenal face. Release of Ca²⁺ and ATP hydrolysis are accompanied by the dephosphorylation that leads to the conformational change back to the E1 state, which represents the end of a Ca²⁺ transport cycle.

In human cells, two different genes, ATP2C1 and ATP2C2, encode four SPCA1 splice variants and SPCA2, respectively. Similar to SERCAs and other P-type ATPases, SPCA isoforms undergoes Ca²⁺ (or Mn²⁺)-binding-induced autophosphorylation of the conserved asparagine (Asp350 in human SPCA1 and Asp351 in SERCA1a). The reversible SPCA cycle transferring ion across the membrane is also facilitated by the conformational change between the E1 and E2 statuses with different affinity to the ion and orientation. Different from SERCAs that transport two Ca²⁺ ions upon the hydrolysis of one ATP, the SPCA pumps contain only one Ca²⁺-binding site, therefore, transport only one Ca²⁺ ion into the Golgi lumen per ATP. Another unique characteristic in contrast to most other Ca²⁺-ATPases is that SPCAs display high affinity toward Mn²⁺ and thus also act as Mn²⁺ transporters. Since only one ion-binding site is conserved in SPCAs, it is suggested that only one Ca²⁺ or Mn²⁺ is transported by the energy derived from the hydrolysis of one ATP molecule. Although both SPCA isoforms catalyze Ca²⁺ or Mn²⁺-dependent autophosphorylation, SPCA1 shows a lower Ca²⁺ affinity and slightly lower Mn²⁺ affinity comparing with SPCA2.

It is widely accepted that SERCAs and SPCAs both contribute to the Ca²⁺ uptake into the Golgi since it is only partially inhibited by the SERCA inhibitor thapsigargin.

TMEM165 was first identified in congenital disorders of glycosylation (CDG) and localize to the Golgi. Loss of TMEM165 function interrupts Ca²⁺, pH, and Mn²⁺ homeostasis and causes glycosylation abnormalities, which are restored by Mn²⁺ supplementation. Therefore, TMEM165 is proposed to be a Golgi-localized Ca²⁺/Mn²⁺ antiporter involved in glycosylation regulation. TMEM165 is relocated to lysosomes and subjected to lysosomal degradation upon high Mn²⁺ exposure in a Rab7 and Rab5 independent manner, indicating a Mn²⁺-dependent TMEM165 quality control mechanism involving direct targeting of the protein to the lysosomal degradation pathway.

In one aspect, the SPCA protein is SPCA1 or SPCA2. In some aspects, increasing SPCA1 or SPCA2 expression and/or activity in the cell comprises contacting the cell with a nucleic acid encoding a SPCA1 or SPCA2 protein. In other aspects, increasing SPCA1 or SPCA2 expression and/or activity in the cell comprises contacting the cell with a vector comprising a nucleic acid encoding a SPCA1 or SPCA2 protein. In many aspects, vector is an adeno-associated virus (AAV) vector, optionally the vector is an AAV2 or an AAV9 vector. In some aspects, increasing SPCA1 or SPCA2 expression and/or activity in the cell comprises contacting the cell with a small molecule agonist or activator of SPCA1 or SPCA2. In one aspect, the cell is a neuronal cell or a skin cell. In some aspects, the neuronal cell is a cell from a subject having a neurodegenerative disease. In various aspects, the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD). In other aspects, the skin cell is a cell from a subject having Hailey Hailey disease (IHID). In some aspects, the cell is from a subject having Brody myopathy (BRM), acrokeratosis verruciformis (AKV), Darier disease (DD), Hailey-Hailey disease (HHD), or a congenital disorder of glycosylation, Type Ilk (CDG2K). In other aspects, the cell is from a subject carrying a mutation is a gene selected from the group consisting of a sarcoplasmic/endoplasmic reticulum calcium ATPase (SERCA), a secretory pathway Ca²⁺-transporting ATPase (SPCA) and TMEM165.

In another embodiment, the invention provides, a method of modulating Golgi ion concentration in a cell including increasing the expression and/or activity of a metal ion transporter in the cell, thereby modulating Golgi ion concentration in the cell.

In one aspect, modulating Golgi ion concentration in the cell includes (i) increasing Ca²⁺ and/or Mn²⁺ concentration in the Golgi, (ii) decreasing Ca²⁺ and/or Mn²⁺ in the cytoplasm, (iii) restoring Golgi Ca²⁺ and/or Mn²⁺ level and/or (iv) increasing cytoplasmic Ca²⁺ and/or Mn²⁺ clearance rate in the cell.

In one aspect, the metal ion transporter is a Ca²⁺/Mn²⁺ transporter. In another aspect, the Ca²⁺/Mn²⁺ transporter is a secretory pathway calcium ATPase (SPCA) protein. In one aspect, the SPCA protein is SPCA1 or SPCA2.

In some aspects, increasing SPCA1 or SPCA2 expression and/or activity in the cell comprises contacting the cell with a nucleic acid encoding a SPCA1 or SPCA2 protein.

As used herein, the term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, in particular, in the form of RNA which can be prepared by in vitro transcription from a DNA template. The RNA can moreover be modified before application by stabilizing sequences, capping, and polyadenylation. The nucleic acid, once expressed in a cell can be translated into a polypeptide or protein of interest.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

In other aspects, increasing SPCA1 expression and/or activity in the cell comprises contacting the cell with a vector comprising a nucleic acid encoding a SPCA1 protein.

The terms “vector”, “expression vector”, and “plasmid DNA” are used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains and the like. In a specific embodiment the proteins of the present invention include a nucleic acid encoding a SPCA1 or SPCA2 protein.

Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver a polynucleotide described herein.

Suitable bacterial vectors for use in practice of the invention methods include pQE70™, pQE60™, pQE-9™, pBLUESCRIPT™ SK, pBLUESCRIPT™ KS, pTRC99a™ pKK223-3™, pDR540™, PAC™ and pRIT2T™. Suitable eukaryotic vectors for use in practice of the invention methods include pWLNEO™, pXTI™, pSG5™, pSVK3™ pBPV™, pMSG™, and pSVLSV40™. Suitable eukaryotic vectors for use in practice of the invention methods include pWLNEO™, pXTI™, pSG5™, pSVK3™, pBPV™, pMSG™ and pSVLSV40™.

One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.”

Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

In many aspects, vector is an adeno-associated virus (AAV) vector.

The term “AAV” is an abbreviation for adeno-associated virus and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, AAVDJ, rhlO, derivatives and hybrids thereof, avian AAV, bovine AAV, canine AAV, equine AAV, primate AAV, non-primate AAV, and ovine AAV. Additionally, any engineered or variant derived from ancestral AAV sequence reconstruction can be used as a vector. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single-stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

Techniques contemplated herein for gene therapy of somatic cells include delivery via a viral vector (e.g., retroviral, adenoviral, AAV, helper-dependent adenoviral systems, hybrid adenoviral systems, herpes simplex, pox virus, lentivirus, and Epstein-Barr virus), and non-viral systems, such as physical systems (naked DNA, DNA bombardment, electroporation, hydrodynamic, ultrasound, and magnetofection), and chemical systems (cationic lipids, different cationic polymers, and lipid polymers).

In some aspects, the vector is an AAV9 vector or an AAV2 vector.

As used herein, the term “AAV9” vector is meant to refer to an AAV9 vector or another AAV vector optimal for neuronal cell transduction.

As used herein, the term “AAV2” vector is meant to refer to an AAV2 vector or another AAV vector optimal for skin cell transduction.

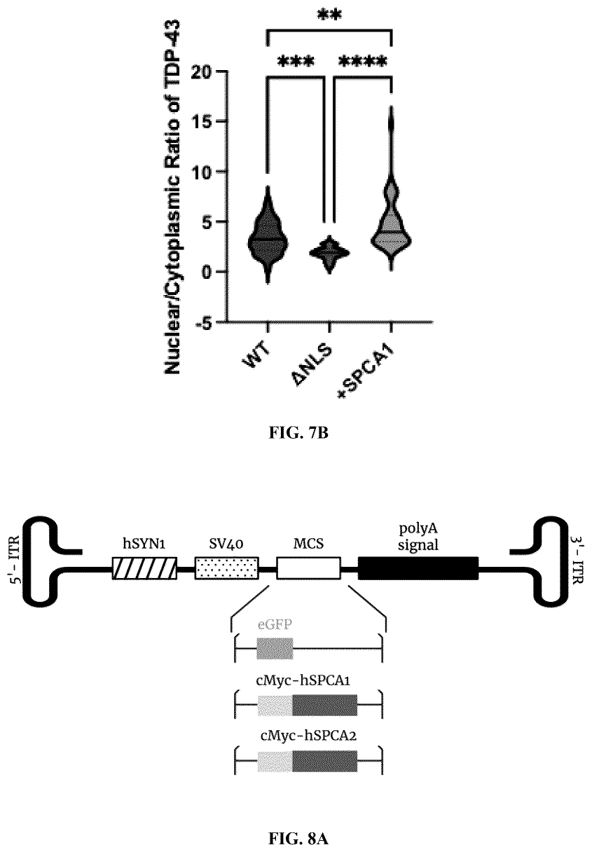

The vector described herein can include various elements, including but not limited to 3′ and 5′untranslated regions (UTRs), a promoter, a transgene, optionally including a tag and/or a polyA signal (see FIG. 8A for exemplary vectors of the invention).

In the viral vectors described herein, regulatory elements controlling transcription can be used, those are generally derived from mammalian, microbial, viral or insect genes. The ability to replicate in a host, usually conferred by an origin of replication, and a selection gene to facilitate recognition of transformants may additionally be incorporated. Those of skill in the art can select a suitable regulatory region to be included in such a vector.

For example, expression vector usually comprises one or more promoters, operably linked to the nucleic acid of interest. As used herein, a promoter is intended to mean a polynucleotide sequence capable of facilitating transcription of genes in operable linkage with the promoter. Several types of promoters are well known in the art and suitable for use with the present invention. The promoter can be constitutive or inducible. Non-limiting examples of constitutive promoters include cytomegalovirus (CMV) promoter and the Rous sarcoma virus promoter, that allows for unregulated expression in mammalian cells. The promoter can be selected based on the tissue selectivity of the promoter. For example, the vector can include a promoter that induces expression of the transgene in neural cells or in skin cells.

In one aspect, the promoter is a human synuclein (hSYN) promoter.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

In various aspects, the nucleic acid construct encodes at least one protein tag. A variety of protein tags are known in the art, such as epitope tags, affinity tags, solubility enhancing tags, and the like. Affinity tags are the most commonly used tag for aiding in protein purification while epitope tags aid in the identification of proteins. One of skill in the art would understand that some tags may be useful as more than one type of tag. Examples of various additional tags and linkers that may be used with the present invention include, haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO), and ubiquitin (Ub).

In one aspect, the vector includes an epitope tag. In various aspects, the epitope tag is a c-Myc epitope tag, including a c-Myc epitope tag comprising the nucleic acid sequence of SEQ ID NO:5.

Methods for attaching two individual elements usually require the use of a linker. The term “linker” as used herein refers to any bond, small molecule, or other vehicle which allows the substrate and the active agent to be targeted to the same area, tissue, or cell, for example by physically linking the individual portions of the conjugate. A linker can be any chemical moiety that is capable of linking a compound, usually a drug, to a cell-binding agent in a stable, covalent manner. Linkers can be susceptible to or be substantially resistant to acid-induced cleavage, light-induced cleavage, peptidase-induced cleavage, esterase-induced cleavage and disulfide bond cleavage at conditions under which the compound or the antibody remains active. Linkers are classified upon their chemical motifs, well known in the art, including disulfide groups, hydrazine or peptides (cleavable), or thioester groups (non-cleavable). Linkers also include charged linkers, and hydrophilic forms thereof as known in the art. Non-limiting examples of linker include glycine rich linker, such as poly(L-Gly), (Poly L-Glycine linkers); poly(L-Glu), (PolyL-Glutamine linkers); poly (L-Lys), (Poly L-Lysine linkers).

In various aspects, the vector includes a Gly-rich linker. In some aspects, the Gly-rich linker includes the nucleic acid sequence of SEQ ID NO: 6.

The vector includes a transgene of interest, operably linked to the promoter. In various aspects, the vector described herein includes a transgene including a SPCA gene. The SPCA gene can be SPCA1a or SPCA2. In many aspects, the SPCA gene is a neuronal splice variant of the gene. In some aspects, the vector includes a transgene including the nucleic acid sequence of SEQ ID NO: 7 or 8.

In some aspects, the vector includes SEQ ID NO:3 or SEQ ID NO:4.

In other aspects, the vector includes SEQ ID NO: 7 or SEQ ID NO: 8. In some aspects, the vector includes SEQ ID NO: 7 or SEQ ID NO: 8 and one or more of SEQ ID NOs5, 6 and 7.

In one aspect the vector includes SEQ ID NO:1 or SEQ ID NO:2.

In some aspects, increasing SPCA1 or SPCA2 expression and/or activity in the cell comprises contacting the cell with a small molecule agonist or activator of SPCA1 or SPCA2.

As used herein, the phrase “small organic molecule” is meant to refer to a biologically active molecule, or a molecule that has a biological effect in a cell. In certain embodiments the active molecule may be an inorganic molecule, an organic molecule, a drug compound, a peptide, a polypeptide, such as an enzyme or transcription factor.

By “agonist” or “activator” it is meant that the small molecule has a positive effect on the expression and/or activity of SPCA1 or SPCA2. That is, a small molecule agonist or activator can act through direct replacement of the physiological molecules or through indirect replacement, by acting on intermediate agent leading to SPCA1 or SPCA2 expression or activation.

The methods described herein rely on the modulation of Golgi ion concentration and of maintaining and/or restoring Golgi integrity in a cell.

The methods described herein can be applied to any cell type. One of skill in the art would readily appreciate that when applied to a method of treatment, for example, the cell type would be related to the disease to be treated. For example, when applied to a method of treating a neurodegenerative disease, the cell type might be neuronal cell; when applied to a method of treating a cutaneous disease, the cell type might be a skin cell, etc.

In one aspect, the cell is a neuronal cell or a skin cell.

“Neuronal cell” or neuron” generally refers to any information carrier cells located within the central nervous system (CNS) or peripheral nervous system (PNS). Neurons can be classified by function, where they can be classified as afferent neurons (or sensory neurons, conveying information from tissues and organs into the central nervous system), efferent neurons (motor neurons, which transmit signals from the central nervous system to the effector cells) or interneurons (which connect neurons within specific regions of the central nervous system).Alternatively, or additionally, neurons can be anatomically characterized as: unipolar (including one single process—exclusively sensory neurons; their dendrites are receiving sensory information, sometimes directly from the stimulus itself), bipolar (including one axon and one dendrite; they are found mainly in the olfactory epithelium, and as part of the retina), multipolar (including one axon and two or more dendrites; include for example pyramidal cells, Purkinje cells, anterior horn cells, and granule cell), anaxonic (where the axon cannot be distinguished from the dendrite(s)) and pseudo-unipolar (including one process which then serves as both an axon and a dendrite).

“Skin cell” as used herein is meant to refer to any cell that can be found in the skin, including cells from the epidermis (keratinocytes, melanocytes, Langerhans cells and Merkel cells), dermis (including mast cells, vascular smooth muscle cells, fibroblasts, and immune cells) and subcutis (including fat cells—adipocytes, nerves and blood vessels cells).

In some aspect, the neuronal cell is a cell from a subject having a neurodegenerative disease.

In various aspects, the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD).

In other aspects, the skin cell is a cell from a subject having Hailey Hailey disease (HHD).

The methods described herein are meant to be applied to cells and subjects in need of at least maintaining and/or restoring Golgi integrity in the cell, and/or modulating ionic concentration in a Golgi lumen in the cell. As detailed above, there are various genes encoding proteins that are involved in such processes. Non-limiting examples of such genes include sarcoplasmic/endoplasmic reticulum calcium ATPase (SERCA), a secretory pathway Ca²⁺-transporting ATPase (SPCA) and TMEM165.

In one aspect, the cell is from a subject carrying a mutation is a gene selected from the group consisting of a sarcoplasmic/endoplasmic reticulum calcium ATPase (SERCA), a secretory pathway Ca²⁺-transporting ATPase (SPCA) and TMEM165.

Mutations in one of those genes are associated with genetic diseases. Therefore, the methods described herein when applied to cells and subjects in need of at least maintaining and/or restoring Golgi integrity in the cell, and/or modulating ionic concentration in a Golgi lumen in the cell can be used for the treatment of diseases and conditions associated with mutations in genes encoding a metal ion transporter, such as a SERCA gene, a SPCA gene or TMEM165. For example, the disease can be Brody myopathy (BRM), acrokeratosis verruciformis (AKV), Darier disease (DD), Hailey-Hailey disease (HHD), or a congenital disorder of glycosylation, Type Ilk (CDG2K). Therefore, in some aspects, the cell is from a subject having BRM, AKV, DD, HHD, or CDG2K.

In another embodiment, the invention provides a method of treating a neurodegenerative disease in subject including increasing the expression and/or activity of a metal ion transporter in the subject, thereby treating the neurodegenerative disease.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

By “increasing the expression and/or activity of a metal ion transporter” it is meant that the methods described herein can induce the expression or activity of a protein having a metal ion transporter activity in cells where said expression or activity is altered. Inducing or increasing said expression and/or activity includes changes in the level of expression and/or activity from any level of change between an initially altered level and a normal/unaltered level, all the way up to a complete restoration of the expression or activity. In cases where the increase (for example of the expression/activity) is restored, it can also be said that the phenotype is reversed.

In one aspect, the metal ion transporter is a Ca²⁺/Mn²⁺ transporter. In another aspect, the Ca²⁺/Mn²⁺ transporter is a secretory pathway calcium ATPase (SPCA) protein.

In one aspect, the SPCA protein is SPCA1 or SPCA2. In one aspect, increasing SPCA1 or SPCA2 expression and/or activity in the subject includes increasing SPCA1 or SPCA2 expression and/or activity in a cell from the subject.

In one aspect, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof. In some aspects, increasing SPCA1 or SPCA2 expression and/or activity in a cell inhibits the progression of the neurological disease and/or inhibits the development of clinical symptoms.

In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector comprising a nucleic acid encoding a SPCA1 or SPCA2 protein. In various aspects, the vector is an AAV vector, optionally the vector is an AAV9 vector.

In some aspects, increasing SPCA1 or SPCA2 expression and/or activity in the cell from the subject includes contacting the cell with a small molecule agonist or activator of SPCA1 or SPCA2.

In another aspect, increasing SPCA1 or SPCA2 expression and/or activity in the subject includes administering to the subject a vector including a nucleic acid encoding a SPCA1 or SPCA2 protein.

The terms “therapeutically effective amount”, “effective dose,” “therapeutically effective dose”, “effective amount,” or the like refer to that amount of the subject agent that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the disease). Such amount should be sufficient to increase SPCA1 or SPCA2 expression and/or activity. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, intracapsular, intraorbital, intracutaneous, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic administration subcuticular, intraarticulare, subcapsular, intrasternal, ocular administrations, as well infusion, and nebulization, or any combination thereof.

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat neurodegenerative diseases and HHD. Specifically, the administration of a nucleic acid encoding a SPCA1 or SPCA2 protein, a small molecule agonist or activator of SPCA1 or SPCA2 or a vector including a nucleic acid encoding a SPCA1 or SPCA2 protein to a subject can be in combination with any other therapeutic regimen known and used for the treatment of any of the diseases including neurodegenerative diseases described herein, and of HHD. Such therapies can be administered prior to, simultaneously with, or following administration of the composition of the present invention. In non-limiting examples, a SPCA1 or SPCA2 therapy (i.e., any of the treatment described herein to be administered to subjects, e.g., a nucleic acid encoding a SPCA1 or SPCA2 protein, a small molecule agonist or activator of SPCA1 or SPCA2, a vector including a nucleic acid encoding a SPCA1 or SPCA2 protein, or a small molecule agonist or activator of SPCA1 or SPCA2) may be administered in combination with an HDAC inhibitor.

In some aspects, the vector is an AAV vector, optionally the vector is an AAV2 or an AAV9 vector.

In one aspect, increasing SPCA1 or SPCA2 expression and/or activity in the subject includes administering to the subject a small molecule agonist or activator of SPCA1 or SPCA2.

In some aspects, the neurodegenerative disease is characterized by Golgi fragmentation in neuron cells, mitochondrial membrane depolarization in neuron cells, increased ROS production in neuron cells, stress body (RNA-protein) formation in neuron cells, dysregulation of Ca²⁺ homeostasis in neuron cells, and/or Mn²⁺ sensitivity in neuron cells.

In other aspects, the neurodegenerative disease is selected from the group consisting of ALS, AD, FTD, HD, CID and PD.

Alzheimer's disease (AD) is a neurodegenerative disease that usually starts slowly and progressively worsens. It is the cause of 60-70% of cases of dementia. The most common early symptom is difficulty in remembering recent events. As the disease advances, symptoms can include problems with language, disorientation (including easily getting lost), mood swings, loss of motivation, self-neglect, and behavioral issues. Gradually, bodily functions are lost, ultimately leading to death. Although the speed of progression can vary, the typical life expectancy following diagnosis is three to nine years. The disease process is largely associated with amyloid plaques, neurofibrillary tangles, and loss of neuronal connections in the brain. A probable diagnosis is based on the history of the illness and cognitive testing with medical imaging and blood tests to rule out other possible causes. Initial symptoms are often mistaken for normal aging. Examination of brain tissue is needed for a definite diagnosis, but this can only take place after death.

Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Degeneration is also present in brainstem nuclei particularly the locus coeruleus in the pons. Studies using MRI and PET have documented reductions in the size of specific brain regions in people with Alzheimer's disease as they progressed from mild cognitive impairment to Alzheimer's disease, and in comparison, with similar images from healthy older adults. Both A3 plaques and neurofibrillary tangles are clearly visible by microscopy in brains of those with Alzheimer's disease, especially in the hippocampus. However, Alzheimer's disease may occur without neurofibrillary tangles in the neocortex. Plaques are dense, mostly insoluble deposits of beta-amyloid peptide and cellular material outside and around neurons. Tangles (neurofibrillary tangles) are aggregates of the microtubule-associated protein tau which has become hyperphosphorylated and accumulate inside the cells themselves. Although many older individuals develop some plaques and tangles as a consequence of aging, the brains of people with Alzheimer's disease have a greater number of them in specific brain regions such as the temporal lobe. Lewy bodies are not rare in the brains of people with Alzheimer's disease.

Amyotrophic lateral sclerosis (ALS), also known as motor neuron disease (MND) or Lou Gehrig's disease, is a neurodegenerative disease that results in the progressive loss of motor neurons that control voluntary muscles. ALS is the most common type of motor neuron disease. Early symptoms of ALS include stiff muscles, muscle twitches, and gradual increasing weakness and muscle wasting. Limb-onset ALS begins with weakness in the arms or legs, while bulbar-onset ALS begins with difficulty speaking or swallowing. Half of the people with ALS develop at least mild difficulties with thinking and behavior, and about 15% develop frontotemporal dementia. Most people experience pain. The muscles affected are responsible for chewing food, speaking, and walking. Motor neuron loss continues until the ability to eat, speak, move, and finally the ability to breathe is lost. ALS eventually causes paralysis and early death, usually from respiratory failure. Most cases of ALS (about 90% to 95%) have no known cause and are known as sporadic ALS. However, both genetic and environmental factors are believed to be involved. The remaining 5% to 10% of cases have a genetic cause linked to a history of the disease in the family, and these are known as familial ALS. About half of these genetic cases are due to one of two specific genes. ALS and frontotemporal dementia (FTD) are considered to be part of a common disease spectrum (ALS-FTD) because of genetic, clinical, and pathological similarities. The underlying mechanism involves damage to both upper and lower motor neurons; in ALS-FTD, neurons in the frontal and temporal lobes of the brain die as well. The diagnosis is based on a person's signs and symptoms, with testing done to rule out other potential causes.

The defining feature of ALS is the death of both upper motor neurons (located in the motor cortex of the brain) and lower motor neurons (located in the brainstem and spinal cord). In ALS with frontotemporal dementia, neurons throughout the frontal and temporal lobes of the brain die as well. The pathological hallmark of ALS is the presence of inclusion bodies (abnormal aggregations of protein) known as Bunina bodies in the cytoplasm of motor neurons. In about 97% of people with ALS, the main component of the inclusion bodies is TDP-43 protein; however, in those with SOD1 or FUS mutations, the main component of the inclusion bodies is SOD1 protein or FUS protein, respectively. The gross pathology of ALS, which are features of the disease that can be seen with the naked eye, include skeletal muscle atrophy, motor cortex atrophy, sclerosis of the corticospinal and corticobulbar tracts, thinning of the hypoglossal nerves (which control the tongue), and thinning of the anterior roots of the spinal cord. Aside from the death of motor neurons, two other characteristics common to most ALS variants are focal initial pathology, meaning that symptoms start in a single spinal cord region, and progressive continuous spread, meaning that symptoms spread to additional regions over time. Prion-like propagation of misfolded proteins from cell to cell may explain why ALS starts in one area and spreads to others. The glymphatic system may also be involved in the pathogenesis of ALS.

Parkinson's disease (PD), or simply Parkinson's, is a long-term degenerative disorder of the central nervous system that mainly affects the motor system. The symptoms usually emerge slowly, and as the disease worsens, non-motor symptoms become more common. The most obvious early symptoms are tremors, rigidity, slowness of movement, and difficulty with walking. Cognitive and behavioral problems may also occur with depression, anxiety, and apathy occurring in many people with PD. Parkinson's disease dementia becomes common in the advanced stages of the disease. Those with Parkinson's can also have problems with their sleep and sensory systems. The motor symptoms of the disease result from the death of cells in the substantia nigra, a region of the midbrain, leading to a dopamine deficit. The cause of this cell death is poorly understood but involves the build-up of misfolded proteins into Lewy bodies in the neurons. Collectively, the main motor symptoms are also known as parkinsonism or parkinsonian syndrome. No cure for PD is known; treatment aims to reduce the effects of the symptoms. Initial treatment is typically with the medications levodopa (L-DOPA), MAO-B inhibitors, or dopamine agonists. As the disease progresses, these medications become less effective, while at the same time producing a side effect marked by involuntary muscle movements. At that time, medications may be used in combination and doses may be increased. Diet and certain forms of rehabilitation have shown some effectiveness at improving symptoms. Surgery to place microelectrodes for deep brain stimulation has been used to reduce motor symptoms in severe cases where drugs are ineffective. The main pathological characteristics of PD are cell death in the brain's basal ganglia (affecting up to 70% of the dopamine-secreting neurons in the substantia nigra pars compacta by the end of life). In Parkinson's disease, alpha-synuclein becomes misfolded and clump together with other alpha-synuclein. Cells are unable to remove these clumps, and the alpha-synuclein becomes cytotoxic, damaging the cells. These clumps can be seen in neurons under a microscope and are called Lewy bodies. Loss of neurons is accompanied by the death of astrocytes (star-shaped glial cells) and a significant increase in the number of microglia (another type of glial cell) in the substantia nigra. Braak staging is a way to explain the progression of the parts of the brain affected by PD. According to this staging, PD starts in the medulla and the olfactory bulb before moving to the substantia nigra pars compacta and the rest of the midbrain/basal forebrain. Movement symptom onset is associated when the disease begins to affect the substantia nigra pars compacta. Brain cells could be lost by several proposed mechanisms. One mechanism consists of an abnormal accumulation of the protein alpha-synuclein bound to ubiquitin in the damaged cells. This insoluble protein accumulates inside neurons forming inclusions called Lewy bodies. According to the Braak staging, a classification of the disease based on pathological findings proposed by Heiko Braak, Lewy bodies first appear in the olfactory bulb, medulla oblongata, and pontine tegmentum; individuals at this stage may be asymptomatic or may have early nonmotor symptoms (such as loss of sense of smell, or some sleep or automatic dysfunction). As the disease progresses, Lewy bodies develop in the substantia nigra, areas of the midbrain and basal forebrain, and finally, the neocortex. These brain sites are the main places of neuronal degeneration in PD, but Lewy bodies may not cause cell death, and they may be protective (with the abnormal protein sequestered or walled off).

Frontotemporal dementia (FTD), or frontotemporal degeneration disease, or frontotemporal neurocognitive disorder, encompasses several types of dementia involving the progressive degeneration of frontal and temporal lobes. FTDs broadly present as behavioral or language disorders with gradual onsets. The three main subtypes or variant syndromes are a behavioral variant (bvFTD) previously known as Pick's disease, and two variants of primary progressive aphasia—semantic variant (svPPA), and nonfluent variant (nfvPPA). Two rare distinct subtypes of FTD are neuronal intermediate filament inclusion disease (NIFID), and basophilic inclusion body disease. Other related disorders include corticobasal syndrome and FTD with amyotrophic lateral sclerosis (ALS) FTD-ALS also called FTD-MND.

Frontotemporal dementias are mostly early-onset syndromes that are linked to frontotemporal lobar degeneration (FTLD), which is characterized by progressive neuronal loss predominantly involving the frontal or temporal lobes, and a typical loss of more than 70% of spindle neurons, while other neuron types remain intact. There are three main histological subtypes found at post-mortem: FTLD-tau, FTLD-TDP, and FTLD-FUS. In rare cases, patients with clinical FTD were found to have changes consistent with Alzheimer's disease on autopsy. The most severe brain atrophy appears to be associated with behavioral variant FTD, and corticobasal degeneration. With regard to the genetic defects that have been found, repeat expansion in the C9orf72 gene is considered a major contribution to frontotemporal lobar degeneration, although defects in the GRN and MAPT genes are also associated with it.

Huntington's disease (HD), also known as Huntington's chorea, is an incurable neurodegenerative disease that is mostly inherited. The earliest symptoms are often subtle problems with mood or mental/psychiatric abilities. A general lack of coordination and an unsteady gait often follow. It is also a basal ganglia disease causing a hyperkinetic movement disorder known as chorea. As the disease advances, uncoordinated, involuntary body movements of chorea become more apparent. Physical abilities gradually worsen until coordinated movement becomes difficult and the person is unable to talk. Mental abilities generally decline into dementia, depression, apathy, and impulsivity at times.

HD is typically inherited from an affected parent, who carries a mutation in the huntingtin gene (HTT). However, up to 10% of cases are due to a new mutation. The huntingtin gene provides the genetic information for huntingtin protein (Htt). Expansion of CAG repeats of cytosine-adenine-guanine (known as a trinucleotide repeat expansion) in the gene coding for the huntingtin protein results in an abnormal mutant protein (mHtt), which gradually damages brain cells through a number of possible mechanisms. The mutant protein is dominant, so having one parent who is a carrier of the trait is sufficient to trigger the disease in their children.

No cure for HD is known, and full-time care is required in the later stages. Treatments can relieve some symptoms and in some, improve quality of life. The best evidence for treatment of the movement problems is with tetrabenazine. HD affects about 4 to 15 in 100,000 people of European descent. Complications such as pneumonia, heart disease, and physical injury from falls reduce life expectancy, although fatal aspiration pneumonia is commonly cited as the ultimate cause of death for those with the condition.

Creutzfeldt-Jakob disease (CJD), also known as subacute spongiform encephalopathy or neurocognitive disorder due to prion disease, is a fatal neurodegenerative disease. Early symptoms include memory problems, behavioral changes, poor coordination, and visual disturbances. Later symptoms include dementia, involuntary movements, blindness, weakness, and coma. About 70% of people die within a year of diagnosis. CJD is caused by a type of abnormal shaping of a protein known as a prion. Infectious prions are misfolded proteins that can cause normally folded proteins to also become misfolded. About 85% of cases of CJD occur for unknown reasons, while about 7.5% of cases are inherited in an autosomal dominant manner. Exposure to brain or spinal tissue from an infected person may also result in spread. There is no evidence that sporadic CID can spread among people via normal contact or blood transfusions, although this is possible in variant Creutzfeldt-Jakob disease. Diagnosis involves ruling out other potential causes. An electroencephalogram, spinal tap, or magnetic resonance imaging may support the diagnosis. There is no specific treatment for CJD. Opioids may be used to help with pain, while clonazepam or sodium valproate may help with involuntary movements. CJD affects about one person per million people per year. It is classified as a type of transmissible spongiform encephalopathy. Inherited CJD accounts for about 10% of prion disease cases. Sporadic CJD is different from bovine spongiform encephalopathy (mad cow disease) and variant Creutzfeldt-Jakob disease (vCJD).

In some aspects, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof.

In other aspects, increasing SPCA1 or SPCA2 expression and/or activity in a cell inhibits the progression of the neurological disease and/or inhibits the development of clinical symptoms.

Among some of the cellular phenotypes described herein when referring to neurodegenerative or cutaneous diseases, Golgi fragmentation, mitochondrial depolarization, ROS production, stress body (RNA-protein) formation, dysregulation of Ca²⁺ homeostasis (ER/Golgi stores) and Mn²⁺ sensitivity are considered early cellular phenotype that precede other cellular phenotypes (such as inclusion bodies, neuromuscular denervation, apoptosis for example) and clinical symptoms. For example, Golgi fragmentation is observed in ˜50% of sporadic ALS cases and in ˜70% of familial ALS cases and is also common to other neurodegenerative disorders (such as AD and PD). By increasing SPCA1 or SPCA2 expression and/or activity in cells, the methods described herein (i) maintains and/or restore Golgi integrity, (ii) decrease Golgi fragmentation, (iii) decrease mitochondrial depolarization, (iv) decrease oxidative stress, (v) decrease ROS production, (vi) increase cell viability, (vii) decrease cholesterol accumulation, (viii) improve sorting, processing and/or secretion and post-translational modification of cargo protein in the cell, (ix) modulate ionic concentration in a Golgi lumen, (x) protect cells against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, and/or (xvi) correcting cytoplasmic mislocalization of TDP-43 in the cell. Without wishing to be bound to any particular theory, it is believed that by limiting the occurrence and/or by restoring some of the early cellular affections occurring in cells before the development of clinical symptoms, the methods described herein can be useful for preventing/inhibiting/delaying the apparition of those clinical symptoms, and therefore can be useful for preventing/delaying the progression of said disease.

In an additional embodiment, the invention provides a method of treating a disease in a subject including increasing the expression and/or activity of a metal ion transporter in the subject, wherein the disease is selected from the group consisting of Brody myopathy (BRM), acrokeratosis verruciformis (AKV), Darier disease (DD), Hailey-Hailey disease (HHD), or a congenital disorder of glycosylation, Type Ilk (CDG2K), thereby treating the disease in the subject.

In one aspect, the metal ion transporter is a Ca²⁺/Mn²⁺ transporter. In another aspect, the Ca²⁺/Mn²⁺ transporter is a secretory pathway calcium ATPase (SPCA) protein. In one aspect, the SPCA protein is SPCA1 or SPCA2. In one aspect, increasing SPCA1 or SPCA2 expression and/or activity in the subject includes increasing SPCA1 or SPCA2 expression and/or activity in a cell from the subject.

In one aspect, increasing SPCA1 or SPCA2 expression and/or activity in the subject includes increasing SPCA1 or SPCA2 expression and/or activity in a cell from the subject. In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector including a nucleic acid encoding a SPCA1 or SPCA2 protein. In various aspects, the vector is an AAV vector, optionally the vector is an AAV2 or an AAV9 vector. In some aspects, increasing SPCA1 or SPCA2 expression and/or activity in the cell from the subject includes contacting the cell with a small molecule agonist or activator of SPCA1 or SPCA2.

In another aspect, increasing SPCA1 or SPCA2 expression and/or activity in the subject includes administering to the subject a vector comprising a nucleic acid encoding a SPCA1 or SPCA2 protein.

Without wishing to limit the administration route in any way, it is noted that topical administration may be a preferred route of administration for the treatment of cutaneous diseases.

In some aspects, the vector is an AAV vector, optionally the vector is an AAV2 or an AAV9 vector.

In one aspect, increasing SPCA1 or SPCA2 expression and/or activity in the subject includes administering to the subject a small molecule agonist or activator of SPCA1 or SPCA2.

In some aspects, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof.

In an additional embodiment, the invention provides a method of treating HHD in a subject comprising increasing the expression and/or activity of a SPCA protein in the subject, thereby treating HHD.

Hailey-Hailey disease (HHD), or familial benign chronic pemphigus or familial benign pemphigus is a genetic disorder that causes blisters to form on the skin. HHD is characterized by outbreaks of rashes and blisters on the skin. Affected areas of skin undergo repeated blistering and inflammation and may be painful to the touch. Areas where the skin folds, as well as the armpits, groin, neck, buttocks and under the breasts are most commonly affected. In addition to blistering, other symptoms which accompany HHD include acantholysis, erythema and hyperkeratosis. It typically begins in late teenage years or in a person's 30s or 40s.

The cause of the disease is a haploinsufficiency of the enzyme ATP2C1. A mutation on one copy of the gene causes the cells of the skin not to adhere together properly due to malformation of intercellular desmosomes, causing acantholysis, blisters and rashes. There is no known cure. Topical steroid preparations often help outbreaks; use of the weakest corticosteroid that is effective is recommended to help prevent thinning of the skin. Drugs such as antibiotics, antifungals, corticosteroids, dapsone, methotrexate, thalidomide, etretinate, cyclosporine and, most recently, intramuscular alefacept may control the disease but are ineffective for severe chronic or relapsing forms of the disease. Intracutaneous injections of botulinum toxin to inhibit perspiration may be of benefit. Maintaining a healthy weight, avoiding heat and friction of affected areas, and keeping the area clean and dry may help prevent flares. Some have found relief in laser resurfacing that burns off the top layer of the epidermis, allowing healthy non-affected skin to regrow in its place. Secondary bacterial, fungal and/or viral infections are common and may exacerbate an outbreak. Some have found that outbreaks are triggered by certain foods, hormone cycles and stress. In many cases naltrexone, taken daily in low doses, appears to help.

In one aspect, the SPCA protein is SPCA1 or SPCA2.

In one aspect, increasing SPCA1 or SPCA2 expression and/or activity in the subject includes increasing SPCA1 or SPCA2 expression and/or activity in a cell from the subject. In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector including a nucleic acid encoding a SPCA1 or SPCA2 protein. In various aspects, the vector is an AAV vector, optionally the vector is an AAV2 vector. In some aspects, increasing SPCA1 or SPCA2 expression and/or activity in the cell from the subject includes contacting the cell with a small molecule agonist or activator of SPCA1 or SPCA2.

In another aspect, increasing SPCA1 or SPCA2 expression and/or activity in the subject includes administering to the subject a vector comprising a nucleic acid encoding a SPCA1 or SPCA2 protein.

Without wishing to limit the administration route in any way, it is noted that topical administration may be a preferred route of administration for the treatment of cutaneous diseases.

In some aspects, the vector is an AAV vector, optionally the vector is an AAV2 vector. In one aspect, increasing SPCA1 or SPCA2 expression and/or activity in the subject includes administering to the subject a small molecule agonist or activator of SPCA1 or SPCA2.

In some aspects, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof.

In an additional embodiment, the invention provides a method of treating amyotrophic lateral sclerosis (ALS) in a subject including increasing the expression and/or activity of a secretory pathway calcium ATPase (SPCA) protein in the subject.

In one aspect, the SPCA protein is SPCA1 or SPCA2.

In one aspect, increasing SPCA1 or SPCA2 expression and/or activity in the subject includes increasing SPCA1 or SPCA2 expression and/or activity in a cell from the subject. In some aspects, increasing SPCA1 or SPCA2 expression and/or activity in the cell from the subject includes contacting the cell with a nucleic acid encoding a SPCA1 or SPCA2 protein. In other aspects, increasing SPCA1 or SPCA2 expression and/or activity in the cell from the subject includes contacting the cell with a vector including a nucleic acid encoding a SPCA1 or SPCA2 protein. In various aspects, the vector is an AAV vector, optionally the vector is an AAV2 vector. In some aspects, increasing SPCA1 or SPCA2 expression and/or activity in the cell from the subject includes contacting the cell with a small molecule agonist or activator of SPCA1 or SPCA2.

In another aspect, increasing SPCA1 or SPCA2 expression and/or activity in the subject includes administering to the subject a vector comprising a nucleic acid encoding a SPCA1 or SPCA2 protein.

Without wishing to limit the administration route in any way, it is noted that topical administration may be a preferred route of administration for the treatment of cutaneous diseases.

In some aspects, the vector is an AAV vector, optionally the vector is an AAV2 vector. In one aspect, increasing SPCA1 or SPCA2 expression and/or activity in the subject includes administering to the subject a small molecule agonist or activator of SPCA1 or SPCA2.

In some aspects, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof.

In one aspect, increasing SPCA1 or SPCA2 expression and/or activity in the cell from the subject includes contacting the cell with a vector comprising a nucleic acid encoding a SPCA1 or SPCA2 protein. In various aspects, the vector is an adeno-associated virus (AAV) vector. In one aspect, the vector is an AAV9 vector.

In various aspects, the AAV vector includes SEQ ID NO: 7, SEQ ID NO:8, SEQ ID NO:1 or SEQ ID NO:2.

In another aspect, treating ALS includes improving hindlimb weakness and paralysis, inhibiting forelimb issues, inhibiting muscle atrophy, increasing grip, reducing tremors improving posture, inhibiting weight loss, and/or inhibiting paralysis in the subject.

In a further embodiment, the invention provides a method of inhibiting TDP-43 cytoplasmic mislocalization, inhibiting TDP-43 cytoplasmic aggregation and/or maintaining TDP-43 nuclear localization in a cell including increasing the expression and/or activity of a metal ion transporter in the cell, thereby inhibiting TDP-43 cytoplasmic mislocalization, inhibiting TDP-43 cytoplasmic aggregation and/or maintaining TDP-43 nuclear localization in the cell.

TAR DNA-binding protein 43 (TDP-43), or transactive response DNA binding protein 43 kDa, is a protein that in humans is encoded by the TARDBP gene. TDP-43 is 414 amino acid residues long that consists of 4 domains: an N-terminal domain spanning residues 1-76 (NTD) with a well-defined fold that has been shown to form a dimer or oligomer; 2 highly conserved folded RNA recognition motifs spanning residues 106-176 (RRM1) and 191-259 (RRM2), respectively, required to bind target RNA and DNA; an unstructured C-terminal domain encompassing residues 274-414 (CTD), which contains a glycine-rich region, is involved in protein-protein interactions, and harbors most of the mutations associated with familial amyotrophic lateral sclerosis. The NTD located between residues 1 and 76 is involved in TDP-43 polymerization. Indeed, dimers are formed by head-to-head interactions between NTDs, and the polymer thus obtained allows for pre-mRNA splicing. However, further oligomerization brings to more toxic accumulations. TDP-43 can aggregate with one another, accumulate, and spread using prion mechanisms of action. TDP-43 polypeptides or aggregates (or TDP-43 prions) are pathological and can be detected in subjects diagnosed with neurodegenerative diseases, associated with the accumulation of pathological protein in neurons, responsible for neurodegeneration.

TDP-43 is a transcriptional repressor that binds to chromosomally integrated TAR DNA and represses HIV-1 transcription. In addition, this protein regulates alternate splicing of the CFTR gene. TDP-43 has been shown to bind both DNA and RNA and have multiple functions in transcriptional repression, pre-mRNA splicing and translational regulation. Transcriptome-wide binding sites characterization revealed that thousands of RNAs are bound by TDP-43 in neurons. TDP-43 was originally identified as a transcriptional repressor that binds to chromosomally integrated trans-activation response element (TAR) DNA and represses HIV-1 transcription. It was also reported to regulate alternate splicing of the CFTR gene and the apoA-II gene. In spinal motor neurons TDP-43 has also been shown in humans to be a low molecular weight neurofilament (hNFL) mRNA-binding protein. It has also shown to be a neuronal activity response factor in the dendrites of hippocampal neurons suggesting possible roles in regulating mRNA stability, transport and local translation in neurons.

TDP-43 protein is a key element of the non-homologous end joining (NHEJ) enzymatic pathway that repairs DNA double-strand breaks (DSBs) in pluripotent stem cell-derived motor neurons. TDP-43 is rapidly recruited to DSBs where it acts as a scaffold for the further recruitment of the XRCC4-DNA ligase protein complex that then acts to seal the DNA breaks. In TDP-43 depleted human neural stem cell-derived motor neurons, as well as in sporadic ALS patients' spinal cord specimens there is significant DSB accumulation and reduced levels of NHEJ.

As used herein, the terms “inhibiting TDP-43 cytoplasmic mislocalization,” “inhibiting TDP-43 cytoplasmic aggregation,” and “maintaining TDP-43 nuclear localization” are meant to refer to TDP-43 localization and TDP-cytoplasmic aggregation in a cell with an increased expression and/or activity of a metal ion transporter in the cell, e.g., in a cell modified to express increased levels of SPCA1 or SPCA2, as compared to a cell without increased expression and/or activity of a metal ion transporter in the cell, e.g., in a cell that is not modified to express increased levels of SPCA1 or SPCA2.

In one aspect, the metal ion transporter is a Ca²⁺/Mn²⁺ transporter. In some aspects, the Ca²⁺/Mn²⁺ transporter is a secretory pathway calcium ATPase (SPCA) protein. In many aspects, the SPCA protein is SPCA1 or SPCA2. In another aspect, increasing SPCA1 or SPCA2 expression and/or activity in the cell includes contacting the cell with a nucleic acid encoding a SPCA1 or SPCA2 protein. In one aspect, increasing SPCA1 or SPCA2 expression and/or activity in the cell includes contacting the cell with a vector comprising a nucleic acid encoding a SPCA1 or SPCA2 protein. In some aspects, the vector is an adeno-associated virus (AAV) vector, optionally wherein the vector is an AAV9 vector. In another aspect, increasing SPCA1 or SPCA2 expression and/or activity in the cell from the subject includes contacting the cell with a small molecule agonist or activator of SPCA1 or SPCA2. In various aspects, increasing SPCA1 or SPCA2 expression and/or activity in a cell includes (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca²⁺ and/or Mn²⁺ concentration in the Golgi in the cell, (xii) decreasing Ca²⁺ and/or Mn²⁺ in the cytoplasm in the cell, (xiii) restoring Golgi Ca²⁺ and/or Mn²⁺ level in the cell, (xiv) increasing cytoplasmic Ca²⁺ and/or Mn²⁺ clearance rate in the cell, (xv) maintaining nuclear localization of TDP-43 in the cell, (xvi) correcting cytoplasmic mislocalization of TDP-43 in the cell, or (xvii) any combination thereof. In many aspects, the cell is a neuronal cell or a skin cell. In some aspects, the neuronal cell is a cell from a subject having a neurodegenerative disease. In various aspects, the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD). In other aspects, the cell is from a subject carrying a mutation is a gene selected from the group consisting of a sarcoplasmic/endoplasmic reticulum calcium ATPase (SERCA), a secretory pathway Ca²⁺-transporting ATPase (SPCA) and TMEM165.

Presented below are examples discussing the effects of the induction of SPCA1 or SPCA2 expression contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Study Design

Golgi fragmentation is an early cellular phenotype observed in about 50% of sporadic cases of Amyotrophic Lateral Sclerosis (ALS) and about 70% of familial cases. Golgi fragmentation precedes other cellular phenotypes such as inclusion bodies, neuromuscular denervation and apoptosis. To evaluate the role of SPCA1 on ALS cellular phenotypes (including Golgi function) motor neurons-like NSC-34 cells were transfected to express either a wild-type copy of SOD1 gene (referred to as SOD1^{WT} cells) or a copy of the SOD1 gene including an ALS mutation (referred to as SOD1^{G93A} cells—an ALS model). Cells transfected with the mutant gene were simultaneously transfected either with empty vector (as control) or with pMF8 plasmid to induce SPCA1 expression (referred to as SOD1^{G93A}+SPCA1 cells) and then differentiated into neurons.

SOD1^{G93A} are considered an ALS model, that is characterized by Golgi fragmentation, mitochondrial depolarization (ROS production), cholesterol accumulation, stress body (RNA-protein) formation, dysregulation of Ca²⁺ homeostasis (ER/Golgi stores) and Mn²⁺ sensitivity.

SOD1^{WT} cells, SOD1^{G93A} cells and SOD1^{G93A}+SPCA1 cells were then assessed to evaluate the impact of the rescue of the mutant phenotype on Golgi fragmentation, mitochondrial depolarization, ROS production and cholesterol accumulation.

### Example 2

### Effect of SPCA1 Expression on Golgi Ion Concentration

Golgi ion concentration was evaluated using NSC-34 neurons transfected with SOD1^{WT}, SOD^{G93A} mutant and SOD1^{G93A} mutant plus SPCA1 (+SPCA1).

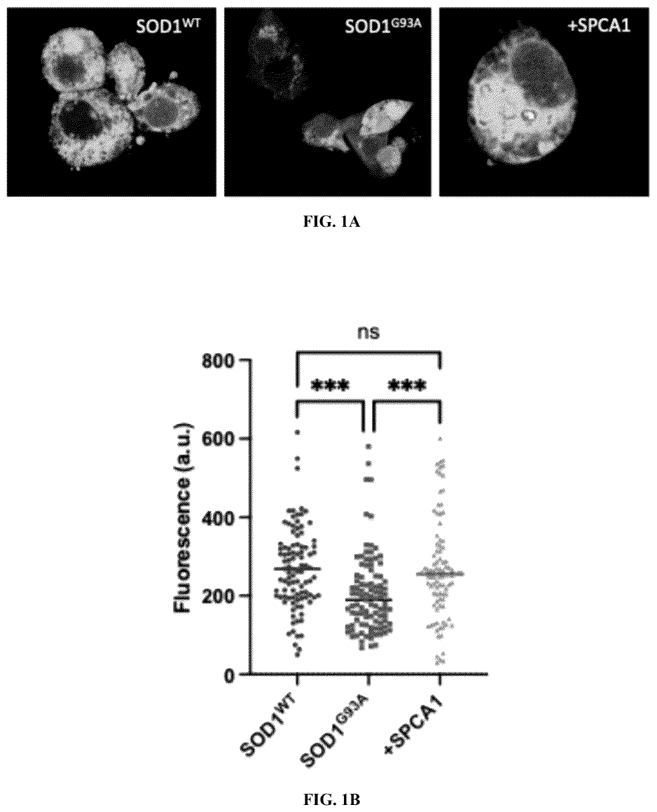

The cells were loaded with the fluorescent Ca²⁺ sensor Mag Fluo-4 which accumulates in the Golgi. As illustrated in FIG. 1A and quantified in FIG. 1B, the experiment illustrates the ability of SPCA1 to modulate ion concentrations by increasing Ca²⁺ in the Golgi.

The cells were then loaded with the fluorescent Ca²⁺ sensor Fluo-8 which accumulates in the cytoplasm. As illustrated in FIG. 1C, SPCA1 accelerates the decrease of Ca²⁺ in the cytoplasm. This is further validated by the analysis of the rate of clearance which was faster in “+SPCA1” cells, as shown by the slope of the traces plotted in FIG. 1D.

### Example 3

### Effect of SPCA1 Expression on Golgi Fragmentation

Golgi fragmentation was evaluated by immunofluorescence, using anti-GOLGIN97 antibodies to detect Golgin subfamily A member 1 (GOLGA1), a protein involved in vesicular trafficking at the Golgi apparatus level and involved in endosome-to-Golgi trafficking.

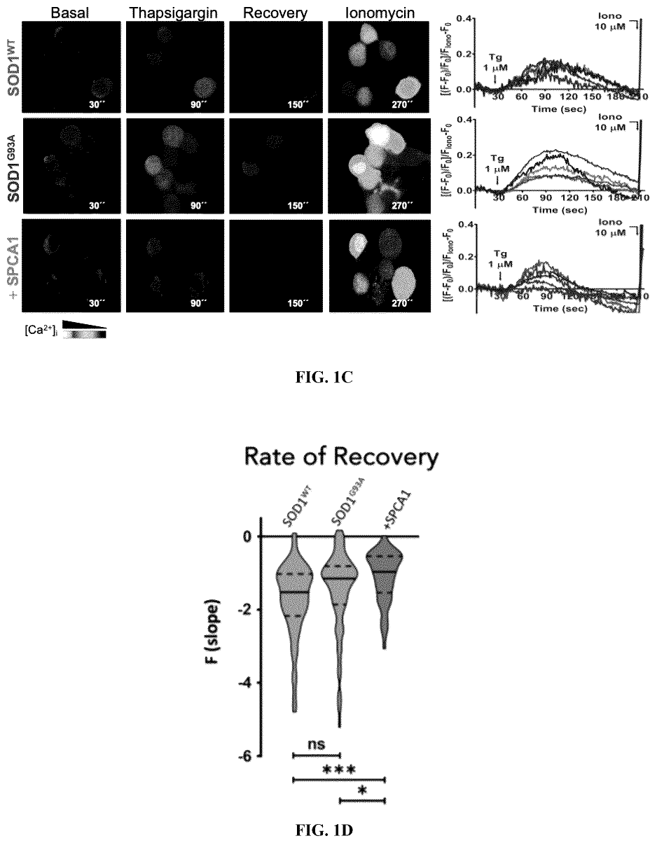

As illustrated in FIGS. 2A and 2B, this example shows that it was found that SOD1^{G93A} cells presented increased levels of Golgi fragmentation as compared to SOD1^{WT} cells, and that inducing or increasing SPCA1 expression in cells reversed Golgi fragmentation, as seen in SOD1^{G93A}+SPCA1 cells.

### Example 4

### Effect of SPCA1 Expression on Mitochondrial Membrane Potential

Mitochondrial membrane potential was measured in SOD1^{WT} cells, SOD1^{G93A} cells and SOD1^{G93A}+SPCA1 cells, as well as in control cells treated with a protonophore (positive control), by staining the cells with MitoTracker® Red CMXROS, and analyzing the cells by flow cytometry. Depolarization of mitochondrial membrane potential appears as a left shift in cell populations when analyzing MitoTracker® Red CMXROS staining levels in a histogram.

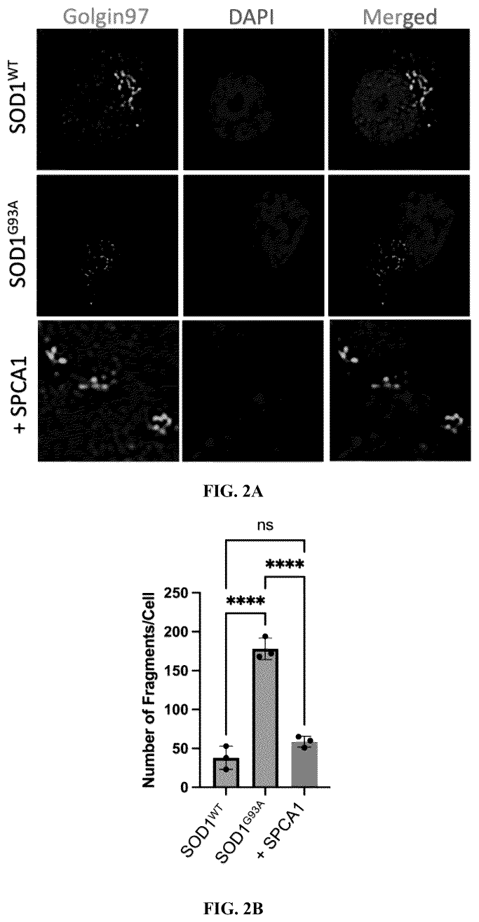

As illustrated in FIG. 3A, mutant cells (SOD1^{G93A}—middle histogram) showed were found depolarized relative to WT control (SOD1^{WT}—top histogram) as seen by a left shift in peak. Mutant cells expressing SPCA1 (SOD1^{G93A}+SPCA1 cells—bottom histogram) showed the highest percent of cells in white box, as compared to both SOD1^{G93A} cells and SOD1^{WT} cells, indicating a reversing in the left shifted peak. Topmost trace in FIG. 3B shows effect of a protonophore on mitochondrial membrane potential, as a positive control.

This example shows that it was found that inducing or increasing SPCA1 expression in cells rescued cells from mitochondrial depolarization.

### Example 5

### Effect of Spca1 Expression on Oxidative Stress

Oxidative stress was measured in cells by staining the NSC-34 differentiated motor neurons with DCHF-DA stain (a fluorogenic dye that measures hydroxyl, peroxyl and other reactive oxygen species (ROS) activity within the cell) and analyzing the cells by flow cytometry.

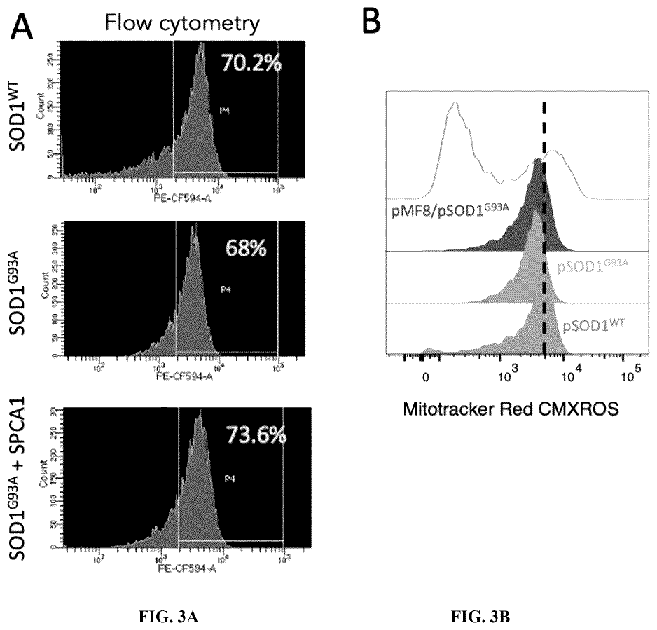

As illustrated in FIGS. 4A and 4B, it was found that as compared to SOD1^{G93A} cells (FIG. 4A), SOD1^{G93A}+SPCA1 cells (FIG. 4B) presented fewer dead cells (8.66% as compared to 13.9%, as well as fewer cells with high levels of ROS (8.39% as compared to 15.8%), and an increase in the number of viable cells (82.8% versus 69.9%).

This example shows that it was found that inducing or increasing SPCA1 expression in cells rescued cells from oxidative stress (see FIG. 4C).

### Example 6

### Effect of SPCA1 Expression on Cholesterol Accumulation

Accumulation of free cholesterol is associated with several neurodegenerative disorders, and was therefore investigated in SOD1^{WT} cells, SOD1^{G93A} cells and SOD1^{G93A}+SPCA1 cells. Deposition/accumulation of cholesterol was measured by immunofluorescence of the cells stained with filipin staining.

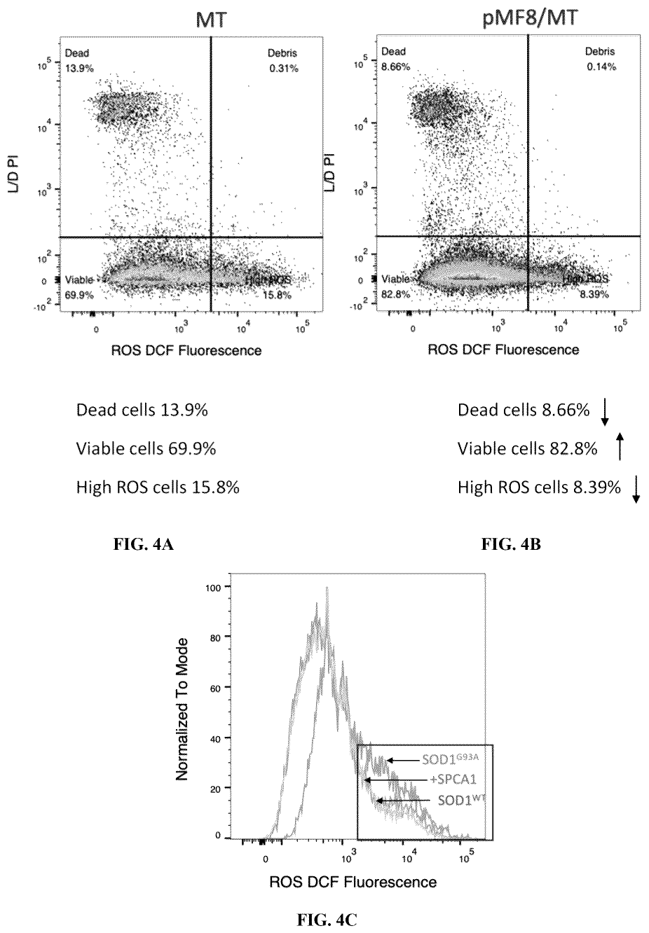

As illustrated in FIG. 5, SOD1^{G93A} cells (middle row) showed significant accumulation of cholesterol as compared to SOD1^{WT} cells (top row).

Expression of SPCA1 in the SOD1 mutant SOD1^{G93A}+SPCA1 cells (bottom row) was found to reduce cholesterol accumulation to levels comparable to wild type levels.

This example shows that it was found that inducing or increasing SPCA1 expression in cells reversed cholesterol accumulation.

### Example 7

### Increasing SPCA1 to Treat Neurodegenerative Diseases

The effects of the induction or increase of SPCA1 expression in cells will be assessed in other ALS cells models, including in patient-derived iPS cells, and in cells presenting different mutations, such as TDP-43 mutations.

The effects of the induction or increase of SPCA1 expression will then be assessed in vivo, in rodent models of ALS, and SPCA1 expression will be induced using AAV vector encoding a SPCA1 gene (AAV-SPCA1 vector).

Alternative or in addition, small molecule agonists/activators for SPCA proteins will be developed and evaluated.

### Example 8

### Increasing SPCA1 to Treat Hailey Hailey Disease (HHD)

Hailey Hailey disease (HHD) is a rare genetic disorder (1:50,000 people) caused by mutations in one copy of SPCA1 (gene ATP2C1). HHD is characterized by painful blisters in skin folds like armpit, neck, and groin that develop fungal infections.

The AAV-SPCA1 vector described in Example 6 will be used to develop AAV-based gene therapy to treat HHD.

The small molecule agonists/activators for SPCA proteins described in Example 6 will be used to develop topical medications to treat HHD.

### Example 9

### Development of a SPCA2 Therapy

The effects observed in the Examples described above with SPCA1 are expected to be reproducible with a SPCA2 therapy.

To assess the exact effects of such a treatment, AAV-SPCA2 vectors will be generated to develop gene therapy to treat HHD. Additionally, or alternatively, small molecule drugs, including histone deacetylase (HDAC) inhibitors (e.g., vorinostat, romidepsin) will be used to increase the expression of SPCA2 in HHD keratinocytes.

Candidate drugs and inhibitors will be contacted with HHD keratinocytes, and the effects will be assessed on the ability to:
maintaining and/or restoring Golgi integrity in the cell,
decreasing Golgi fragmentation in the cell,
decreasing mitochondrial depolarization in the cell,
decreasing oxidative stress in the cell,
decreasing ROS production in the cell,
increasing cell viability,
decreasing cholesterol accumulation in the cell,
improving sorting, processing and/or secretion and post-translational modification of cargo protein in the cell,
modulating ionic concentration in a Golgi lumen in the cell,
protecting the cell against damage, (xi) increasing Ca²⁺ and/or Mn²⁺ concentration in the Golgi in the cell, (xii) decreasing Ca²⁺ and/or Mn²⁺ in the cytoplasm in the cell,
restoring Golgi Ca²⁺ and/or Mn²⁺ level in the cell,
increasing cytoplasmic Ca²⁺ and/or Mn²⁺ clearance rate in the cell,
maintaining nuclear localization of TDP-43 in the cell,
correcting cytoplasmic mislocalization of TDP-43 in the cell.

### Example 10

### Effect of SPCA2 Expression on Nuclear Localization of TDP-43

SOD1^{G93A} cells were modified to express SPCA1 or SPCA2 constructs; SOD1^{WT} cells were used as control. Plasmids expressing hSPCA1 and hSPCA2 were introduced into the SOD1^{G93A} mutant cells, and the effect of SPCA1/SPCA2 expression on TDP-43 localization was assessed.

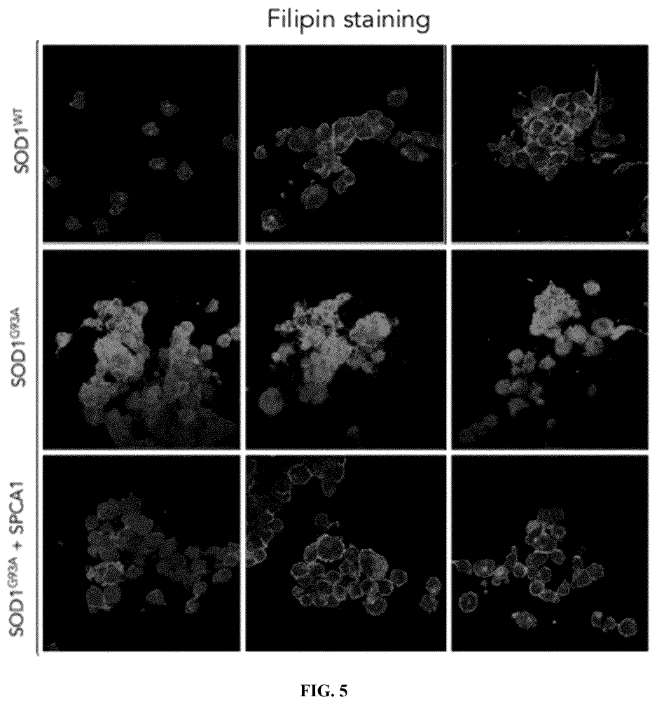

As illustrated in FIG. 6, where the elongation of mitochondria in cells expressing SPCA2 can be observed. The top panel shows representative transmission electron microscopy images and bottom panel shows confocal fluorescence microscopy images of cells labeled with Mitotracker Green. NSC-34 cells expressing either SOD1^{WT} or SOD1^{G93A} plasmids are indicated.

TDP43 (TAR DNA-binding protein 43) is a protein that plays a critical role in amyotrophic lateral sclerosis (ALS), a neurodegenerative disease. In ALS, TDP43 is found in abnormal clumps or aggregates within nerve cells, particularly in the spinal cord and brain. This mislocalization and aggregation of TDP43 is a hallmark of the disease and is believed to contribute to the death of motor neurons, leading to the symptoms of ALS. In FIG. 6, the effect of boosting hSPCA1 expression in NSC-34 cells expressing normal human TDP43 (TDP43 WT) or a mutant with a N-terminal truncation of the first 85 amino acids was assessed, extending into the nuclear localization signal (TDP43 ANLS) that mislocalizes in the cytoplasm. TDP43 is localized by immunofluorescence using an antibody that detects both mouse (endogenous) and human (transfected) TDP43 in NSC-34 cells.

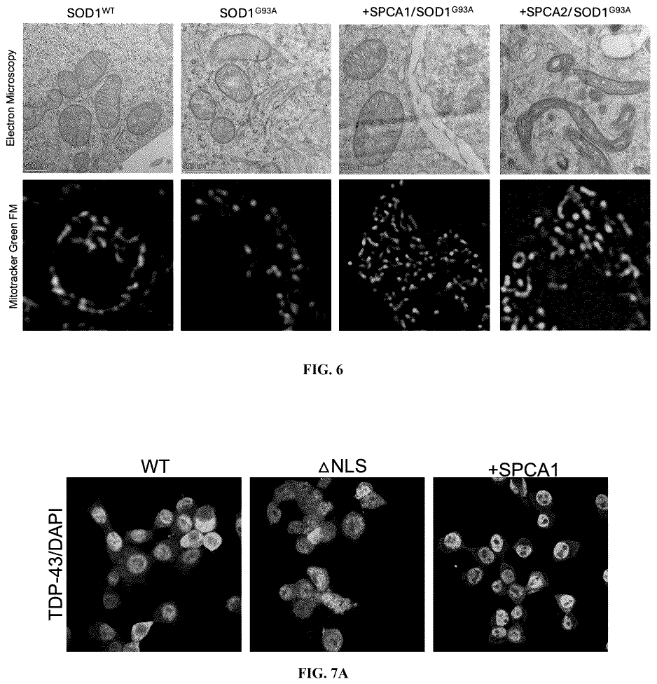

FIGS. 7A-7B illustrate that boosting SPCA1 expression corrects TDP-43 mislocalization.

Two new AAV9 vectors were developed for transgenic expression of myc-tagged SPCA1 and SPCA2 in neurons. In FIG. 8A a schematic of the AAV9 constructs is illustrated. In FIG. 8B shows the use of the developed SPCA1 AAV9 vector to reverse Golgi fragmentation, as evaluated by comparison of immunofluorescence images of the Golgi marker protein Golgin 97 in SOD1^{WT} cells, SOD1^{G93A} cells, and SOD1^{G93A}+SPCA1 AAV9 vector cells.

### Example 11

### Expression of Spca1 in Mouse Motor Cortex Before and After Viral Infection

To assess the efficacy of the induction of the expression of SCPA1 in motor neuron, purified AAV9-SPCA1 virus (SEQ ID NO: 1) was injected unilaterally into the motor cortex of P1 neonate transgenic SOD1-G93A mice, a mouse model of ALS. All pups survived post-injection and appeared unaffected. Pups were sacrificed at 2 weeks, and fixed brain cryosections were immunostained for SPCA1 expression evaluation.

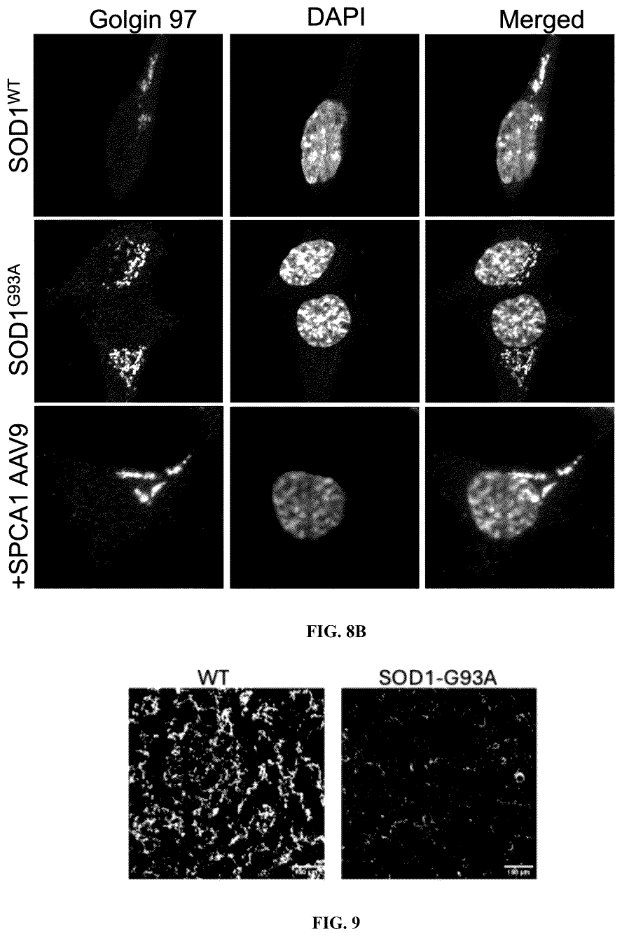

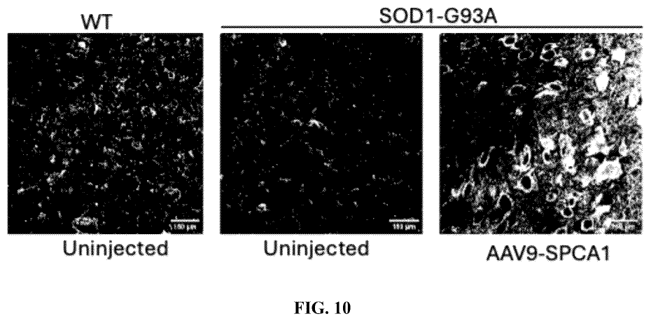

As illustrated in FIG. 10, immunofluorescence sections of mouse motor cortex stained for SPCA1 protein showed reduced expression in SOD1-G93A, compared to the wild type (WT) control in 2-week-old male uninjected mice (see FIG. 10 left and middle panels). This unexpected, reduced expression of SPCA1 expression in the motor cortex of ALS animals was confirmed in older animals as old as 4-month-old male mice (see FIG. 9).

As further illustrated in FIG. 10, immunofluorescence sections of mouse motor cortex stained for SPCA1 protein showed increased SPCA1 expression in the AAV9-SPCA1 injected versus uninjected brain halves of a SOD1-G93A mouse.

### Example 12

### Functional Assessment of the Effect of SPCA1 Rescue In Vivo

Using AAV9 viral gene delivery of GFP (vector control) or SPCA1 (proposed ALS therapy) into the cerebrospinal fluid in mice transgene expression in the brain and spinal cord will be assessed. Disease endpoints will be evaluated using behavioral and histopathological analysis. Both intra-cerebroventricular (ICV; P0) and intrathecal (IT; P40) injections will be evaluated to maximize upper and lower motor neuron transduction.

The AAV9 serotype in conjunction with hSyn1 promoter will ensure motor neuron tropism and selective expression. To assess selective motor neuron transduction, co-staining GFP or SPCA1 (anti-Myc antibody) with Tuj1 and ChAT (motor neuron identity) and GFAP (glia) will be used.

Viral genome copy number will be determined by qPCR.

Markers of inflammation will be evaluated.

Following euthanasia, motor cortex, spinal cord, sciatic nerve and neuromuscular junctions will be examined for histopathological evidence of disease.

SOD1-G93A mice start to show disease pathology at 4 months, beginning with hindlimb weakness and paralysis, progressing to forelimb issues, muscle atrophy, poor grip, tremors, hunched posture, weight loss, and eventually paralysis.

Motor function using rotarod (average latency to drop), fore limb grip strength, and open field tests (center/periphery activity, rearing) will be evaluated to assess the efficacy of the treatment to improve characteristic complication of ALS disease.

SEQUENCES
pAAV-hSyn1-Myc-SPCA1a
SEQ ID NO: 1
ttgagatcctttttttctgcgcgtaatctgctgcttgcaaaaaaaaaacc
accgctaccagcggtggtttgtttgccggatcaagagctaccaactcttt
ttccgaaggtaactggcttcagcagagcgcagataccaaatactgttctt
ctagtgtagccgtagttaggccaccacttcaagaactctgtagcaccgcc
tacatacctcgctctgctaatcctgttaccagtggctgctgccagtggcg
ataagtcgtgtcttaccgggttggactcaagacgatagttaccggataag
gcgcagcggtcgggctgaacggggggttcgtgcacacagcccagcttgga
gcgaacgacctacaccgaactgagatacctacagcgtgagctatgagaaa
gcgccacgcttcccgaagggagaaaggcggacaggtatccggtaagcggc
agggtcggaacaggagagcgcacgagggagcttccagggggaaacgcctg
gtatctttatagtcctgtcgggtttcgccacctctgacttgagcgtcgat
ttttgtgatgctcgtcaggggggcggagcctatggaaaaacgccagcaac
gcggcctttttacggttcctggccttttgctggccttttgctcacatgtc
ctgcaggcagctgcgcgctcgctcgctcactgaggccgcccgggcgtcgg
gcgacctttggtcgcccggcctcagtgagcgagcgagcgcgcagagaggg
agtggccaactccatcactaggggttcctgcggcctaaggcaattgacta
caaaccgagtatctgcagagggccctgcgtatgagtgcaagtgggtttta
ggaccaggatgaggcgggggggggtgcctacctgacgaccgaccccgacc
cactggacaagcacccaacccccattccccaaattgcgcatcccctatca
gagagggggaggggaaacaggatgcggcgaggcgcgtgcgcactgccagc
ttcagcaccgcggacagtgccttcgcccccgcctggcggcgcgcgccacc
gccgcctcagcactgaaggcgcgctgacgtcactcgccggtcccccgcaa
actccccttcccggccaccttggtcgcgtccgcgccgccgccggcccagc
cggaccgcaccacgcgaggcgcgagataggggggcacgggcgcgaccatc
tgcgctgcggcgccggcgactcagcgctgcctcagtctgcggtgggcagc
ggaggagtcgtgtcgtgcctgagagcgcagctgtgctcctgggcaccgcg
cagtccgcccccgcggctcctggccagaccacccctaggaccccctgccc
caagtcgcagccttcgagctagaaaggaaccaattcagtcgacgctagca
agcttggtaccggatccgaattccccgaaccgacagtcggtctcttcacc
aaggccattcgcgccaccatggagcaaaagctcatttctgaggaagatct
caatggtggtggtggtgggttaattaagaaggttgcacgttttcaaaaaa
tacctaatggtgaaaatgagacaatgattcctgtattgacatcaaaaaaa
gcaagtgaattaccagtcagtgaagttgcaagcattctccaagctgatct
tcagaatggtctaaacaaatgtgaagttagtcataggcgagcctttcatg
gctggaatgagtttgatattagtgaagatgagccactgtggaagaagtat
atttctcagtttaaaaatccccttattatgctgcttctggcttctgcagt
catcagtgttttaatgcatcagtttgatgatgccgtcagtatcactgtgg
caatacttatcgttgttacagttgcctttgttcaggaatatcgttcagaa
aaatctcttgaagaattgagtaaacttgtgccaccagaatgccattgtgt
gcgtgaaggaaaattggagcatacacttgcccgagacttggttccaggtg
atacagtttgcctttctgttggggatagagttcctgctgacttacgcttg
tttgaggctgtggatctttccattgatgagtccagcttgacaggtgagac
aacgccttgttctaaggtgacagctcctcagccagctgcaactaatggag
atcttgcatcgagaagtaacattgcctttatgggaacactggtcagatgt
ggcaaagcaaagggtgttgtcattggaacaggagaaaattctgaatttgg
ggaggtttttaaaatgatgcaagcagaagaggcaccaaaaacccctctgc
agaagagcatggacctcttaggaaaacaactttccttttactcctttggt
ataataggaatcatcatgttggttggctggttactgggaaaagatatcct
ggaaatgtttactattagtgtaagtttggctgtagcagcaattcctgaag
gtctccccattgtggtcacagtgacgctagctcttggtgttatgagaatg
gtgaagaaaagggccattgtgaaaaagctgcctattgttgaaactctggg
ctgctgtaatgtgatttgttcagataaaactggaacactgacgaagaatg
aaatgactgttactcacatatttacttcagatggtctgcatgctgaggtt
actggagttggctataatcaatttggggaagtgattgttgatggtgatgt
tgttcatggattctataacccagctgttagcagaattgttgaggcgggct
gtgtgtgcaatgatgctgtaattagaaacaatactctaatggggaagcca
acagaaggggccttaattgctcttgcaatgaagatgggtcttgatggact
tcaacaagactacatcagaaaagctgaatacccttttagctctgagcaaa
agtggatggctgttaagtgtgtacaccgaacacagcaggacagaccagag
atttgttttatgaaaggtgcttacgaacaagtaattaagtactgtactac
ataccagagcaaagggcagaccttgacacttactcagcagcagagagatg
tgtaccaacaagagaaggcacgcatgggctcagcgggactcagagttctt
gctttggcttctggtcctgaactgggacagctgacatttcttggcttggt
gggaatcattgatccacctagaactggtgtgaaagaagctgttacaacac
tcattgcctcaggagtatcaataaaaatgattactggagattcacaggag
actgcagttgcaatcgccagtcgtctgggattgtattccaaaacttccca
gtcagtctcaggagaagaaatagatgcaatggatgttcagcagctttcac
aaatagtaccaaaggttgcagtattttacagagctagcccaaggcacaag
atgaaaattattaagtcgctacagaagaacggttcagttgtagccatgac
aggagatggagtaaatgatgcagttgctctgaaggctgcagacattggag
ttgcgatgggccagactggtacagatgtttgcaaagaggcagcagacatg
atcctagtggatgatgattttcaaaccataatgtctgcaatcgaagaggg
taaagggatttataataacattaaaaatttcgttagattccagctgagca
cgagtatagcagcattaactttaatctcattggctacattaatgaacttt
cctaatcctctcaatgccatgcagattttgtggatcaatattattatgga
tggacccccagctcagagccttggagtagaaccagtggataaagatgtca
ttcgtaaacctcctcgcaactggaaagacagcattttgactaaaaacttg
atacttaaaatacttgtttcatcaataatcattgtttgtgggactttgtt
tgtcttctggcgtgagctacgagacaatgtgattacacctcgagacacaa
caatgaccttcacatgctttgtgttttttgacatgttcaatgcactaagt
tccagatcccagaccaagtctgtgtttgagattggactctgcagtaatag
aatgttttgctatgcagttcttggatccatcatgggacaattactagtta
tttactttcctccgcttcagaaggtttttcagactgagagcctaagcata
ctggatctgttgtttcttttgggtctcacctcatcagtgtgcatagtggc
agaaattataaagaaggttgaaaggagcagggaaaagatccagaagcatg
ttagttcgacatcatcatcttttcttgaagtatgagcggccgcctcgagt
ctagacgactgtgccttctagttgccagccatctgttgtttgcccctccc
ccgtgccttccttgaccctggaaggtgccactcccactgtcctttcctaa
taaaatgaggaaattgcatcgcattgtctgagtaggtgtcattctattct
ggggggtggggggggcaggacagcaagggggaggattgggaagacaatag
caggcatgctggggatgcggtgggctctatggccgcgggccgcaggaacc
cctagtgatggagttggccactccctctctgcgcgctcgctcgctcactg
aggccgggcgaccaaaggtcgcccgacgcccgggctttgcccgggggcct
cagtgagcgagcgagcgcgcagctgcctgcaggggcgcctgatgcggtat
tttctccttacgcatctgtgcggtatttcacaccgcatacgtcaaagcaa
ccatagtacgcgccctgtagcggcgcattaagcgcggcgggtgtggtggt
tacgcgcagcgtgaccgctacacttgccagcgccttagcgcccgctcctt
tcgctttcttcccttcctttctcgccacgttcgccggctttccccgtcaa
gctctaaatcgggggctccctttagggttccgatttagtgctttacggca
cctcgaccccaaaaaacttgatttgggtgatggttcacgtagtgggccat
cgccctgatagacggtttttcgccctttgacgttggagtccacgttcttt
aatagtggactcttgttccaaactggaacaacactcaactctatctcggg
ctattcttttgatttataagggattttgccgatttcggtctattggttaa
aaaatgagctgatttaacaaaaatttaacgcgaattttaacaaaatatta
acgtttacaattttatggtgcactctcagtacaatctgctctgatgccgc
atagttaagccagccccgacacccgccaacacccgctgacgcgccctgac
gggcttgtctgctcccggcatccgcttacagacaagctgtgaccgtctcc
gggagctgcatgtgtcagaggttttcaccgtcatcaccgaaacgcgcgag
acgaaagggcctcgtgatacgcctatttttataggttaatgtcatgataa
taatggtttcttagacgtcaggtggcacttttcggggaaatgtgcgcgga
acccctatttgtttatttttctaaatacattcaaatatgtatccgctcat
gagacaataaccctgataaatgcttcaataatattgaaaaaggaagagta
tgagtattcaacatttccgtgtcgcccttattcccttttttgcggcattt
tgccttcctgtttttgctcacccagaaacgctggtgaaagtaaaagatgc
tgaagatcagttgggtgcacgagtgggttacatcgaactggatctcaaca
gcggtaagatccttgagagttttcgccccgaagaacgttttccaatgatg
agcacttttaaagttctgctatgtggcgcggtattatcccgtattgacgc
cgggcaagagcaactcggtcgccgcatacactattctcagaatgacttgg
ttgagtactcaccagtcacagaaaagcatcttacggatggcatgacagta
agagaattatgcagtgctgccataaccatgagtgataacactgcggccaa
cttacttctgacaacgatcggaggaccgaaggagctaaccgcttttttgc
acaacatgggggatcatgtaactcgccttgatcgttgggaaccggagctg
aatgaagccataccaaacgacgagcgtgacaccacgatgcctgtagcaat
ggcaacaacgttgcgcaaactattaactggcgaactacttactctagctt
cccggcaacaattaatagactggatggaggcggataaagttgcaggacca
cttctgcgctcggcccttccggctggctggtttattgctgataaatctgg
agccggtgagcgtgggtctcgcggtatcattgcagcactggggccagatg
gtaagccccccgtatcgtagttatctacacgacggggagtcaggcaacta
tggatgaacgaaatagacagatcgctgagataggtgcctcactgattaag
cattggtaactgtcagaccaagtttactcatatatactttagattgattt
aaaacttcatttttaatttaaaaggatctaggtgaagatcctttttgata
atctcatgaccaaaatcccttaacgtgagttttcgttccactgagcgtca
gaccccgtagaaaagatcaaaggatcttc
pAAV-hSyn1-Myc-SPCA2
SEQ ID NO: 2
ttgagatcctttttttctgcgcgtaatctgctgcttgcaaacaaaaaaac
caccgctaccagcggtggtttgtttgccggatcaagagctaccaactctt
tttccgaaggtaactggcttcagcagagcgcagataccaaatactgttct
tctagtgtagccgtagttaggccaccacttcaagaactctgtagcaccgc
ctacatacctcgctctgctaatcctgttaccagtggctgctgccagtggc
gataagtcgtgtcttaccgggttggactcaagacgatagttaccggataa
ggcgcagcggtcgggctgaacggggggttcgtgcacacagcccagcttgg
agcgaacgacctacaccgaactgagatacctacagcgtgagctatgagaa
agcgccacgcttcccgaagggagaaaggcggacaggtatccggtaagcgg
cagggtcggaacaggagagcgcacgagggagcttccagggggaaacgcct
ggtatctttatagtcctgtcgggtttcgccacctctgacttgagcgtcga
tttttgtgatgctcgtcaggggggcggagcctatggaaaaacgccagcaa
cgcggcctttttacggttcctggccttttgctggccttttgctcacatgt
cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctaaggcaattgact
acaaaccgagtatctgcagagggccctgcgtatgagtgcaagtgggtttt
aggaccaggatgaggcgggggggggtgcctacctgacgaccgaccccgac
ccactggacaagcacccaacccccattccccaaattgcgcatcccctatc
agagagggggaggggaaacaggatgcggcgaggcgcgtgcgcactgccag
cttcagcaccgcggacagtgccttcgcccccgcctggcggcgcgcgccac
cgccgcctcagcactgaaggcgcgctgacgtcactcgccggtcccccgca
aactccccttcccggccaccttggtcgcgtccgcgccgccgccggcccag
ccggaccgcaccacgcgaggcgcgagataggggggcacgggcgcgaccat
ctgcgctgcggcgccggcgactcagcgctgcctcagtctgcggtgggcag
cggaggagtcgtgtcgtgcctgagagcgcagctgtgctcctgggcaccgc
gcagtccgcccccgcggctcctggccagaccacccctaggaccccctgcc
ccaagtcgcagccttcgagctagaaaggaaccaattcagtcgacgctagc
aagcttggtaccggatccgaattccccgaaccgacagtcggtctcttcac
caaggccattcgcgccaccatggagcaaaagctcatttctgaggaagatc
tcaatggtggtggtggtgggttaattaacgtcgagggacgcgtctccgag
ttcctgaagaaactcggcttctcggggggggccgccagtaccaggcgctg
gagaaggacgaagaggaagccttgattgatgaacagagtgagctgaaagc
catcgagaaagagaagaaggtgacagccctgccccccaaggaagcgtgca
aatgccagaaagaggatttggccagagcgttttgtgtggacttacacact
gggctgtcggagttctcggtgacgcagcgccggctggcccatggctggaa
tgagtttgttgctgacaacagcgaacctgtgtggaagaaatacctggatc
agtttaagaaccccctgatcctgctgctgctgggctctgccctggtgagt
gtcctcaccaaggagtatgaggacgccgtcagcatcgccacggcagtgct
tgtcgtggtcactgtcgccttcatccaggagtacaggtcggagaaatctc
tggaagagctgaccaagatggttcctccagaatgtaactgcctaagagaa
ggaaaactccagcacctgcttgctcgagaactggttcctggtgatgtcgt
atctctctcgatcggagaccggatccctgcagacatccgactcactgagg
tcacggacctcttggtggatgaatccagtttcaccggggaagccgagcca
tgtagtaaaacagacagccccttgacaggcggtggggacctcaccaccct
cagcaacatcgtcttcatggggaccctggtgcagtatgggaggggccagg
gggtcgtgattggaacaggggaaagctctcagttcggagaagtgtttaag
atgatgcaggctgaagagacacctaaaactcctttgcagaaaagcatgga
caggctaggaaagcaactgacactcttctcctttggcataatcggtctca
tcatgctcattggctggtcgcaagggaaacaactcctgagtatgttcacg
atcggggtcagcctggctgtggcggctattccagagggtctgcccatcgt
cgtcatggtgacgctggtcctgggagtgctgcggatggccaagaagcggg
tcatcgtgaagaagttacccatcgtggagactttaggttgctgcagcgtt
ctctgttctgacaagacggggactctgactgccaatgaaatgacagtgac
ccagcttgtaacgtcagatgggcttcgtgccgaggtcagcggagttgggt
atgacggtcaagggactgtgtgtcttctaccatccaaggaagtcattaag
gaattttccaatgtctcagtgggaaagttagtggaggcgggctgtgttgc
caacaatgcggtcatcagaaagaacgccgtgatggggcagcccaccgagg
gtgcattgatggccctggcgatgaagatggacttaagtgatattaaaaat
tcatatataagaaaaaaagagattccattcagttcagagcagaagtggat
ggcggtgaaatgcagtctgaagactgaggatcaggaagacatttacttca
tgaaaggggccttggaagaggtgatccgctactgcaccatgtacaacaac
gggggcatccccctgccgctgacgccccagcagaggtcattctgcctgca
ggaagagaagaggatggggtcgctcggtttgcgggtgctggccctggctt
ctgggcccgagctggggcggctgacgtttctaggtcttgtgggcatcatt
gaccccccgagagttggcgtgaaggaagcagtccaggttctctccgagtc
tggtgtgtctgtgaagatgataacgggggatgccctggagacggccttgg
ccataggaagaaacatcggcctgtgcaacgggaagctgcaagccatgtcc
ggggaggaggtggacagcgtggagaagggcgagctggccgaccgcgtggg
gaaggtgtccgtgttcttcaggaccagcccaaagcacaagctcaaaatca
tcaaggctctgcaggagtcaggggcgatcgtggccatgactggggatggg
gtgaacgacgcagtggccctgaagtctgcagacattgggatcgccatggg
gcagacagggacggacgtcagcaaagaggccgccaacatgatcctggtgg
atgatgacttctcagccatcatgaatgcagtggaggaaggcaagggtatt
ttttacaacatcaaaaactttgtccgattccagctgagcacgagcatctc
cgccctgagtctcatcactctgtccaccgtgttcaacctgcccagccccc
tcaacgccatgcagatcctatggatcaacatcatcatggatgggccaccg
gcgcagagcttgggggtagagcccgttgacaaagacgccttcaggcagcc
accacggagtgtgcgggacaccatcctcagcagagccctcatcctgaaga
tcctcatgtccgcggccatcatcatcagcgggaccctctttatcttctgg
aaggagatgcctgaagacagagcaagcactccccgcaccacgacgatgac
gttcacttgttttgtgtttttcgatctcttcaacgccttgacctgccgct
ctcagaccaagctgatatttgagatcggctttctcaggaaccacatgttc
ctctactccgtcctggggtccatcctggggcagctggcggtcatttacat
ccccccgctgcagagggtcttccagacggagaacctgggagcgcttgatt
tgctgtttttaactggattggcctcatccgtcttcattttgtcagagctc
ctcaaactatgtgaaaaatactgttgcagccccaagagagtccagatgca
ccctgaagatgtgtaggcggccgcctcgagtctagacgactgtgccttct
agttgccagccatctgttgtttgcccctcccccgtgccttccttgaccct
ggaaggtgccactcccactgtcctttcctaataaaatgaggaaattgcat
cgcattgtctgagtaggtgtcattctattctggggggtggggggggcagg
acagcaagggggaggattgggaagacaatagcaggcatgctggggatgcg
gtgggctctatggccgcgggccgcaggaacccctagtgatggagttggcc
actccctctctgcgcgctcgctcgctcactgaggccgggcgaccaaaggt
cgcccgacgcccgggctttgcccgggcggcctcagtgagcgagcgagcgc
gcagctgcctgcaggggcgcctgatgcggtattttctccttacgcatctg
tgcggtatttcacaccgcatacgtcaaagcaaccatagtacgcgccctgt
agcggcgcattaagcgcggcgggtgtggtggttacgcgcagcgtgaccgc
tacacttgccagcgccttagcgcccgctcctttcgctttcttcccttcct
ttctcgccacgttcgccggctttccccgtcaagctctaaatcgggggctc
cctttagggttccgatttagtgctttacggcacctcgaccccaaaaaact
tgatttgggtgatggttcacgtagtgggccatcgccctgatagacggttt
ttcgccctttgacgttggagtccacgttctttaatagtggactcttgttc
caaactggaacaacactcaactctatctcgggctattcttttgatttata
agggattttgccgatttcggtctattggttaaaaaatgagctgatttaac
aaaaatttaacgcgaattttaacaaaatattaacgtttacaattttatgg
tgcactctcagtacaatctgctctgatgccgcatagttaagccagccccg
acacccgccaacacccgctgacgcgccctgacgggcttgtctgctcccgg
catccgcttacagacaagctgtgaccgtctccgggagctgcatgtgtcag
aggttttcaccgtcatcaccgaaacgcgcgagacgaaagggcctcgtgat
acgcctatttttataggttaatgtcatgataataatggtttcttagacgt
caggtggcacttttcggggaaatgtgcgcggaacccctatttgtttattt
ttctaaatacattcaaatatgtatccgctcatgagacaataaccctgata
aatgcttcaataatattgaaaaaggaagagtatgagtattcaacatttcc
gtgtcgcccttattcccttttttgcggcattttgccttcctgtttttgct
cacccagaaacgctggtgaaagtaaaagatgctgaagatcagttgggtgc
acgagtgggttacatcgaactggatctcaacagcggtaagatccttgaga
gttttcgccccgaagaacgttttccaatgatgagcacttttaaagttctg
ctatgtggcgcggtattatcccgtattgacgccgggcaagagcaactcgg
tcgccgcatacactattctcagaatgacttggttgagtactcaccagtca
cagaaaagcatcttacggatggcatgacagtaagagaattatgcagtgct
gccataaccatgagtgataacactgcggccaacttacttctgacaacgat
cggaggaccgaaggagctaaccgcttttttgcacaacatgggggatcatg
taactcgccttgatcgttgggaaccggagctgaatgaagccataccaaac
gacgagcgtgacaccacgatgcctgtagcaatggcaacaacgttgcgcaa
actattaactggcgaactacttactctagcttcccggcaacaattaatag
actggatggaggcggataaagttgcaggaccacttctgcgctcggccctt
ccggctggctggtttattgctgataaatctggagccggtgagcgtgggtc
tcgcggtatcattgcagcactggggccagatggtaagccccccgtatcgt
agttatctacacgacggggagtcaggcaactatggatgaacgaaatagac
agatcgctgagataggtgcctcactgattaagcattggtaactgtcagac
caagtttactcatatatactttagattgatttaaaacttcatttttaatt
taaaaggatctaggtgaagatcctttttgataatctcatgaccaaaatcc
cttaacgtgagttttcgttccactgagcgtcagaccccgtagaaaagatc
aaaggatcttc
cMyc-SPCAla
SEQ ID NO: 3
atggagcaaaagctcatttctgaggaagatctcaatggtggtggtggtgg
gttaattaagaaggttgcacgttttcaaaaaatacctaatggtgaaaatg
agacaatgattcctgtattgacatcaaaaaaagcaagtgaattaccagtc
agtgaagttgcaagcattctccaagctgatcttcagaatggtctaaacaa
atgtgaagttagtcataggcgagcctttcatggctggaatgagtttgata
ttagtgaagatgagccactgtggaagaagtatatttctcagtttaaaaat
ccccttattatgctgcttctggcttctgcagtcatcagtgttttaatgca
tcagtttgatgatgccgtcagtatcactgtggcaatacttatcgttgtta
cagttgcctttgttcaggaatatcgttcagaaaaatctcttgaagaattg
agtaaacttgtgccaccagaatgccattgtgtgcgtgaaggaaaattgga
gcatacacttgcccgagacttggttccaggtgatacagtttgcctttctg
ttggggatagagttcctgctgacttacgcttgtttgaggctgtggatctt
tccattgatgagtccagcttgacaggtgagacaacgccttgttctaaggt
gacagctcctcagccagctgcaactaatggagatcttgcatcgagaagta
acattgcctttatgggaacactggtcagatgtggcaaagcaaagggtgtt
gtcattggaacaggagaaaattctgaatttggggaggtttttaaaatgat
gcaagcagaagaggcaccaaaaacccctctgcagaagagcatggacctct
taggaaaacaactttccttttactcctttggtataataggaatcatcatg
ttggttggctggttactgggaaaagatatcctggaaatgtttactattag
tgtaagtttggctgtagcagcaattcctgaaggtctccccattgtggtca
cagtgacgctagctcttggtgttatgagaatggtgaagaaaagggccatt
gtgaaaaagctgcctattgttgaaactctgggctgctgtaatgtgatttg
ttcagataaaactggaacactgacgaagaatgaaatgactgttactcaca
tatttacttcagatggtctgcatgctgaggttactggagttggctataat
caatttggggaagtgattgttgatggtgatgttgttcatggattctataa
cccagctgttagcagaattgttgaggcgggctgtgtgtgcaatgatgctg
taattagaaacaatactctaatggggaagccaacagaaggggccttaatt
gctcttgcaatgaagatgggtcttgatggacttcaacaagactacatcag
aaaagctgaatacccttttagctctgagcaaaagtggatggctgttaagt
gtgtacaccgaacacagcaggacagaccagagatttgttttatgaaaggt
gcttacgaacaagtaattaagtactgtactacataccagagcaaagggca
gaccttgacacttactcagcagcagagagatgtgtaccaacaagagaagg
cacgcatgggctcagcgggactcagagttcttgctttggcttctggtcct
gaactgggacagctgacatttcttggcttggtgggaatcattgatccacc
tagaactggtgtgaaagaagctgttacaacactcattgcctcaggagtat
caataaaaatgattactggagattcacaggagactgcagttgcaatcgcc
agtcgtctgggattgtattccaaaacttcccagtcagtctcaggagaaga
aatagatgcaatggatgttcagcagctttcacaaatagtaccaaaggttg
cagtattttacagagctagcccaaggcacaagatgaaaattattaagtcg
ctacagaagaacggttcagttgtagccatgacaggagatggagtaaatga
tgcagttgctctgaaggctgcagacattggagttgcgatgggccagactg
gtacagatgtttgcaaagaggcagcagacatgatcctagtggatgatgat
tttcaaaccataatgtctgcaatcgaagagggtaaagggatttataataa
cattaaaaatttcgttagattccagctgagcacgagtatagcagcattaa
ctttaatctcattggctacattaatgaactttcctaatcctctcaatgcc
atgcagattttgtggatcaatattattatggatggacccccagctcagag
ccttggagtagaaccagtggataaagatgtcattcgtaaacctcctcgca
actggaaagacagcattttgactaaaaacttgatacttaaaatacttgtt
tcatcaataatcattgtttgtgggactttgtttgtcttctggcgtgagct
acgagacaatgtgattacacctcgagacacaacaatgaccttcacatgct
ttgtgttttttgacatgttcaatgcactaagttccagatcccagaccaag
tctgtgtttgagattggactctgcagtaatagaatgttttgctatgcagt
tcttggatccatcatgggacaattactagttatttactttcctccgcttc
agaaggtttttcagactgagagcctaagcatactggatctgttgtttctt
ttgggtctcacctcatcagtgtgcatagtggcagaaattataaagaaggt
tgaaaggagcagggaaaagatccagaagcatgttagttcgacatcatcat
cttttcttgaagtatga
cMyc-SPCA2
SEQ ID NO: 4
Atggagcaaaagctcatttctgaggaagatctcaatggtggtggtggtgg
gttaattaacgtcgagggacgcgtctccgagttcctgaagaaactcggct
tctcggggggggccgccagtaccaggcgctggagaaggacgaagaggaag
ccttgattgatgaacagagtgagctgaaagccatcgagaaagagaagaag
gtgacagccctgccccccaaggaagcgtgcaaatgccagaaagaggattt
ggccagagcgttttgtgtggacttacacactgggctgtcggagttctcgg
tgacgcagcgccggctggcccatggctggaatgagtttgttgctgacaac
agcgaacctgtgtggaagaaatacctggatcagtttaagaaccccctgat
cctgctgctgctgggctctgccctggtgagtgtcctcaccaaggagtatg
aggacgccgtcagcatcgccacggcagtgcttgtcgtggtcactgtcgcc
ttcatccaggagtacaggtcggagaaatctctggaagagctgaccaagat
ggttcctccagaatgtaactgcctaagagaaggaaaactccagcacctgc
ttgctcgagaactggttcctggtgatgtcgtatctctctcgatcggagac
cggatccctgcagacatccgactcactgaggtcacggacctcttggtgga
tgaatccagtttcaccggggaagccgagccatgtagtaaaacagacagcc
ccttgacaggcggtggggacctcaccaccctcagcaacatcgtcttcatg
gggaccctggtgcagtatgggaggggccagggggtcgtgattggaacagg
ggaaagctctcagttcggagaagtgtttaagatgatgcaggctgaagaga
cacctaaaactcctttgcagaaaagcatggacaggctaggaaagcaactg
acactcttctcctttggcataatcggtctcatcatgctcattggctggtc
gcaagggaaacaactcctgagtatgttcacgatcggggtcagcctggctg
tggcggctattccagagggtctgcccatcgtcgtcatggtgacgctggtc
ctgggagtgctgcggatggccaagaagcgggtcatcgtgaagaagttacc
catcgtggagactttaggttgctgcagcgttctctgttctgacaagacgg
ggactctgactgccaatgaaatgacagtgacccagcttgtaacgtcagat
gggcttcgtgccgaggtcagcggagttgggtatgacggtcaagggactgt
gtgtcttctaccatccaaggaagtcattaaggaattttccaatgtctcag
tgggaaagttagtggaggcgggctgtgttgccaacaatgcggtcatcaga
aagaacgccgtgatggggcagcccaccgagggtgcattgatggccctggc
gatgaagatggacttaagtgatattaaaaattcatatataagaaaaaaag
agattccattcagttcagagcagaagtggatggcggtgaaatgcagtctg
aagactgaggatcaggaagacatttacttcatgaaaggggccttggaaga
ggtgatccgctactgcaccatgtacaacaacgggggcatccccctgccgc
tgacgccccagcagaggtcattctgcctgcaggaagagaagaggatgggg
tcgctcggtttgcgggtgctggccctggcttctgggcccgagctggggcg
gctgacgtttctaggtcttgtgggcatcattgaccccccgagagttggcg
tgaaggaagcagtccaggttctctccgagtctggtgtgtctgtgaagatg
ataacgggggatgccctggagacggccttggccataggaagaaacatcgg
cctgtgcaacgggaagctgcaagccatgtccggggaggaggtggacagcg
tggagaagggcgagctggccgaccgcgtggggaaggtgtccgtgttcttc
aggaccagcccaaagcacaagctcaaaatcatcaaggctctgcaggagtc
aggggcgatcgtggccatgactggggatggggtgaacgacgcagtggccc
tgaagtctgcagacattgggatcgccatggggcagacagggacggacgtc
agcaaagaggccgccaacatgatcctggtggatgatgacttctcagccat
catgaatgcagtggaggaaggcaagggtattttttacaacatcaaaaact
ttgtccgattccagctgagcacgagcatctccgccctgagtctcatcact
ctgtccaccgtgttcaacctgcccagccccctcaacgccatgcagatcct
atggatcaacatcatcatggatgggccaccggcgcagagcttgggggtag
agcccgttgacaaagacgccttcaggcagccaccacggagtgtgcgggac
accatcctcagcagagccctcatcctgaagatcctcatgtccgcggccat
catcatcagcgggaccctctttatcttctggaaggagatgcctgaagaca
gagcaagcactccccgcaccacgacgatgacgttcacttgttttgtgttt
ttcgatctcttcaacgccttgacctgccgctctcagaccaagctgatatt
tgagatcggctttctcaggaaccacatgttcctctactccgtcctggggt
ccatcctggggcagctggcggtcatttacatccccccgctgcagagggtc
ttccagacggagaacctgggagcgcttgatttgctgtttttaactggatt
ggcctcatccgtcttcattttgtcagagctcctcaaactatgtgaaaaat
actgttgcagccccaagagagtccagatgcaccctgaagatgtgtag
cMyc epitope
SEQ ID NO: 5
gagcaaaagctcatttctgaggaagatctc
Gly-rich linker
SEQ ID NO: 6
aatggtggtggtggtgggttaattaag
SPCAla gene
SEQ ID NO: 7
aaggttgcacgttttcaaaaaatacctaatggtgaaaatgagacaatgat
tcctgtattgacatcaaaaaaagcaagtgaattaccagtcagtgaagttg
caagcattctccaagctgatcttcagaatggtctaaacaaatgtgaagtt
agtcataggcgagcctttcatggctggaatgagtttgatattagtgaaga
tgagccactgtggaagaagtatatttctcagtttaaaaatccccttatta
tgctgcttctggcttctgcagtcatcagtgttttaatgcatcagtttgat
gatgccgtcagtatcactgtggcaatacttatcgttgttacagttgcctt
tgttcaggaatatcgttcagaaaaatctcttgaagaattgagtaaacttg
tgccaccagaatgccattgtgtgcgtgaaggaaaattggagcatacactt
gcccgagacttggttccaggtgatacagtttgcctttctgttggggatag
agttcctgctgacttacgcttgtttgaggctgtggatctttccattgatg
agtccagcttgacaggtgagacaacgccttgttctaaggtgacagctcct
cagccagctgcaactaatggagatcttgcatcgagaagtaacattgcctt
tatgggaacactggtcagatgtggcaaagcaaagggtgttgtcattggaa
caggagaaaattctgaatttggggaggtttttaaaatgatgcaagcagaa
gaggcaccaaaaacccctctgcagaagagcatggacctcttaggaaaaca
actttccttttactcctttggtataataggaatcatcatgttggttggct
ggttactgggaaaagatatcctggaaatgtttactattagtgtaagtttg
gctgtagcagcaattcctgaaggtctccccattgtggtcacagtgacgct
agctcttggtgttatgagaatggtgaagaaaagggccattgtgaaaaagc
tgcctattgttgaaactctgggctgctgtaatgtgatttgttcagataaa
actggaacactgacgaagaatgaaatgactgttactcacatatttacttc
agatggtctgcatgctgaggttactggagttggctataatcaatttgggg
aagtgattgttgatggtgatgttgttcatggattctataacccagctgtt
agcagaattgttgaggcgggctgtgtgtgcaatgatgctgtaattagaaa
caatactctaatggggaagccaacagaaggggccttaattgctcttgcaa
tgaagatgggtcttgatggacttcaacaagactacatcagaaaagctgaa
tacccttttagctctgagcaaaagtggatggctgttaagtgtgtacaccg
aacacagcaggacagaccagagatttgttttatgaaaggtgcttacgaac
aagtaattaagtactgtactacataccagagcaaagggcagaccttgaca
cttactcagcagcagagagatgtgtaccaacaagagaaggcacgcatggg
ctcagcgggactcagagttcttgctttggcttctggtcctgaactgggac
agctgacatttcttggcttggtgggaatcattgatccacctagaactggt
gtgaaagaagctgttacaacactcattgcctcaggagtatcaataaaaat
gattactggagattcacaggagactgcagttgcaatcgccagtcgtctgg
gattgtattccaaaacttcccagtcagtctcaggagaagaaatagatgca
atggatgttcagcagctttcacaaatagtaccaaaggttgcagtatttta
cagagctagcccaaggcacaagatgaaaattattaagtcgctacagaaga
acggttcagttgtagccatgacaggagatggagtaaatgatgcagttgct
ctgaaggctgcagacattggagttgcgatgggccagactggtacagatgt
ttgcaaagaggcagcagacatgatcctagtggatgatgattttcaaacca
taatgtctgcaatcgaagagggtaaagggatttataataacattaaaaat
ttcgttagattccagctgagcacgagtatagcagcattaactttaatctc
attggctacattaatgaactttcctaatcctctcaatgccatgcagattt
tgtggatcaatattattatggatggacccccagctcagagccttggagta
gaaccagtggataaagatgtcattcgtaaacctcctcgcaactggaaaga
cagcattttgactaaaaacttgatacttaaaatacttgtttcatcaataa
tcattgtttgtgggactttgtttgtcttctggcgtgagctacgagacaat
gtgattacacctcgagacacaacaatgaccttcacatgctttgtgttttt
tgacatgttcaatgcactaagttccagatcccagaccaagtctgtgtttg
agattggactctgcagtaatagaatgttttgctatgcagttcttggatcc
atcatgggacaattactagttatttactttcctccgcttcagaaggtttt
tcagactgagagcctaagcatactggatctgttgtttcttttgggtctca
cctcatcagtgtgcatagtggcagaaattataaagaaggttgaaaggagc
agggaaaagatccagaagcatgttagttcgacatcatcatcttttcttga
agta
ATP2C2 gene
SEQ ID NO: 8
gtcgagggacgcgtctccgagttcctgaagaaactcggcttctcgggggg
ggccgccagtaccaggcgctggagaaggacgaagaggaagccttgattga
tgaacagagtgagctgaaagccatcgagaaagagaagaaggtgacagccc
tgccccccaaggaagcgtgcaaatgccagaaagaggatttggccagagcg
ttttgtgtggacttacacactgggctgtcggagttctcggtgacgcagcg
ccggctggcccatggctggaatgagtttgttgctgacaacagcgaacctg
tgtggaagaaatacctggatcagtttaagaaccccctgatcctgctgctg
ctgggctctgccctggtgagtgtcctcaccaaggagtatgaggacgccgt
cagcatcgccacggcagtgcttgtcgtggtcactgtcgccttcatccagg
agtacaggtcggagaaatctctggaagagctgaccaagatggttcctcca
gaatgtaactgcctaagagaaggaaaactccagcacctgcttgctcgaga
actggttcctggtgatgtcgtatctctctcgatcggagaccggatccctg
cagacatccgactcactgaggtcacggacctcttggtggatgaatccagt
ttcaccggggaagccgagccatgtagtaaaacagacagccccttgacagg
cggtggggacctcaccaccctcagcaacatcgtcttcatggggaccctgg
tgcagtatgggaggggccagggggtcgtgattggaacaggggaaagctct
cagttcggagaagtgtttaagatgatgcaggctgaagagacacctaaaac
tcctttgcagaaaagcatggacaggctaggaaagcaactgacactcttct
cctttggcataatcggtctcatcatgctcattggctggtcgcaagggaaa
caactcctgagtatgttcacgatcggggtcagcctggctgtggcggctat
tccagagggtctgcccatcgtcgtcatggtgacgctggtcctgggagtgc
tgcggatggccaagaagcgggtcatcgtgaagaagttacccatcgtggag
actttaggttgctgcagcgttctctgttctgacaagacggggactctgac
tgccaatgaaatgacagtgacccagcttgtaacgtcagatgggcttcgtg
ccgaggtcagcggagttgggtatgacggtcaagggactgtgtgtcttcta
ccatccaaggaagtcattaaggaattttccaatgtctcagtgggaaagtt
agtggagggggctgtgttgccaacaatgcggtcatcagaaagaacgccgt
gatggggcagcccaccgagggtgcattgatggccctggcgatgaagatgg
acttaagtgatattaaaaattcatatataagaaaaaaagagattccattc
agttcagagcagaagtggatggcggtgaaatgcagtctgaagactgagga
tcaggaagacatttacttcatgaaaggggccttggaagaggtgatccgct
actgcaccatgtacaacaacgggggcatccccctgccgctgacgccccag
cagaggtcattctgcctgcaggaagagaagaggatggggtcgctcggttt
gcgggtgctggccctggcttctgggcccgagctggggcggctgacgtttc
taggtcttgtgggcatcattgaccccccgagagttggcgtgaaggaagca
gtccaggttctctccgagtctggtgtgtctgtgaagatgataacggggga
tgccctggagacggccttggccataggaagaaacatcggcctgtgcaacg
ggaagctgcaagccatgtccggggaggaggtggacagcgtggagaagggc
gagctggccgaccgcgtggggaaggtgtccgtgttcttcaggaccagccc
aaagcacaagctcaaaatcatcaaggctctgcaggagtcaggggcgatcg
tggccatgactggggatggggtgaacgacgcagtggccctgaagtctgca
gacattgggatcgccatggggcagacagggacggacgtcagcaaagaggc
cgccaacatgatcctggtggatgatgacttctcagccatcatgaatgcag
tggaggaaggcaagggtattttttacaacatcaaaaactttgtccgattc
cagctgagcacgagcatctccgccctgagtctcatcactctgtccaccgt
gttcaacctgcccagccccctcaacgccatgcagatcctatggatcaaca
tcatcatggatgggccaccggcgcagagcttgggggtagagcccgttgac
aaagacgccttcaggcagccaccacggagtgtgcgggacaccatcctcag
cagagccctcatcctgaagatcctcatgtccgcggccatcatcatcagcg
ggaccctctttatcttctggaaggagatgcctgaagacagagcaagcact
ccccgcaccacgacgatgacgttcacttgttttgtgtttttcgatctctt
caacgccttgacctgccgctctcagaccaagctgatatttgagatcggct
ttctcaggaaccacatgttcctctactccgtcctggggtccatcctgggg
cagctggcggtcatttacatccccccgctgcagagggtcttccagacgga
gaacctgggagcgcttgatttgctgtttttaactggattggcctcatccg
tcttcattttgtcagagctcctcaaactatgtgaaaaatactgttgcagc
cccaagagagtccagatgcaccctgaagatgtg

Although the invention has been described with reference to the above examples, it will be understood that modifications and variations are encompassed within the spirit and scope of the invention. Accordingly, the invention is limited only by the following claims.

What is claimed is:

1. A method of maintaining and/or restoring Golgi integrity in a cell comprising increasing the expression and/or activity of a metal ion transporter in the cell, thereby maintaining and/or restoring Golgi integrity in the cell.

2. The method of claim 1, wherein the metal ion transporter is a Ca2+/Mn2+ transporter.

3. The method of claim 2, wherein the Ca²⁺/Mn²⁺ transporter is a secretory pathway calcium ATPase (SPCA) protein.

4. The method of claim 3, wherein the SPCA protein is SPCA1 or SPCA2.

5. The method of claim 4, wherein increasing SPCA1 or SPCA2 expression and/or activity in the cell comprises contacting the cell with a vector comprising a nucleic acid encoding a SPCA1 or SPCA2 protein.

6. The method of claim 5, wherein the vector is an adeno-associated virus (AAV) vector, optionally wherein the vector is an AAV9 vector or an AAV2 vector.

7. The method of claim 6, wherein the AAV vector comprises SEQ ID NO: 7, SEQ ID NO:8, SEQ ID NO:1 or SEQ ID NO:2.

8. The method of claim 4, wherein increasing SPCA1 or SPCA2 expression and/or activity in the cell comprises contacting the cell with a small molecule agonist or activator of SPCA1 or SPCA2.

9. A method of modulating Golgi ion concentration in a cell comprising increasing the expression and/or activity of a metal ion transporter in the cell, thereby modulating Golgi ion concentration in the cell.

10. The method of claim 9, wherein modulating Golgi ion concentration in the cell comprises (i) increasing Ca2+ and/or Mn2+ concentration in the Golgi, (ii) decreasing Ca2+ and/or Mn2+ in the cytoplasm, (iii) restoring Golgi Ca2+ and/or Mn2+ level and/or (iv) increasing cytoplasmic Ca2+ and/or Mn2+ clearance rate in the cell.

11. A method of treating a neurodegenerative disease in subject comprising increasing the expression and/or activity of a metal ion transporter in the subject, thereby treating the neurodegenerative disease.

12. The method of claim 11, wherein increasing the expression and/or activity of a metal ion transporter in the subject comprises increasing SPCA1 or SPCA2 expression and/or activity in a cell from the subject.

13. The method of claim 12, wherein increasing SPCA1 or SPCA2 expression and/or activity in a cell comprises (i) maintaining and/or restoring Golgi integrity in the cell, (ii) decreasing Golgi fragmentation in the cell, (iii) decreasing mitochondrial depolarization in the cell, (iv) decreasing oxidative stress in the cell, (v) decreasing ROS production in the cell, (vi) increasing cell viability, (vii) decreasing cholesterol accumulation in the cell, (viii) improving sorting, processing and/or secretion and post-translational modification of cargo protein in the cell, (ix) modulating ionic concentration in a Golgi lumen in the cell, (x) protecting the cell against damage, (xi) increasing Ca2+ and/or Mn2+ concentration in the Golgi in the cell, (xii) decreasing Ca2+ and/or Mn2+ in the cytoplasm in the cell, (xiii) restoring Golgi Ca2+ and/or Mn2+ level in the cell, (xiv) increasing cytoplasmic Ca2+ and/or Mn2+ clearance rate in the cell, (xv) maintaining nuclear localization of TDP-43 in the cell, (xvi) correcting cytoplasmic mislocalization of TDP-43 in the cell, or (xvii) any combination thereof.

14. The method of claim 12, wherein increasing SPCA1 or SPCA2 expression and/or activity in the cell from the subject comprises contacting the cell with a vector comprising a nucleic acid encoding a SPCA1 or SPCA2 protein.

15. The method of claim 14, wherein the vector is an adeno-associated virus (AAV) vector, optionally wherein the vector is an AAV9 vector.

16. The method of claim 15, wherein the AAV vector comprises SEQ ID NO: 7, SEQ ID NO:8, SEQ ID NO:1 or SEQ ID NO:2.

17. The method of claim 1, wherein the neurodegenerative disease is selected from the group consisting of amyotrophic lateral sclerosis (ALS), Alzheimer's disease (AD), frontotemporal dementia (FTD), Huntington's disease (HD), Creutzfeldt-Jakob disease (CJD) and Parkinson's disease (PD).

18. A method of treating amyotrophic lateral sclerosis (ALS), in a subject comprising increasing the expression and/or activity of a secretory pathway calcium ATPase (SPCA) protein in the subject, thereby treating ALS.

19. The method of claim 18, wherein increasing SPCA1 or SPCA2 expression and/or activity in the cell from the subject comprises contacting the cell with a vector comprising a nucleic acid encoding a SPCA1 or SPCA2 protein, wherein the vector is an adeno-associated virus (AAV) vector.

20. The method of claim 19, wherein the vector is an AAV9 vector.

21. The method of claim 19, wherein the AAV vector comprises SEQ ID NO: 7, SEQ ID NO:8, SEQ ID NO:1 or SEQ ID NO:2.

22. The method of claim 18, wherein treating ALS comprises improving hindlimb weakness and paralysis, inhibiting forelimb issues, inhibiting muscle atrophy, increasing grip, reducing tremors, improving posture, inhibiting weight loss, and/or inhibiting paralysis in the subject.


SEQUENCE LISTING (8 sequences)

SEQ ID NO: 1 - DNA, 6634 bp, synthetic construct
  source 1..6634: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctaaggcaattgactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccttcgagctagaaaggaaccaattcagtcgacgctagcaagcttggtaccggatccgaattccccgaaccgacagtcggtctcttcaccaaggccattcgcgccaccatggagcaaaagctcatttctgaggaagatctcaatggtggtggtggtgggttaattaagaaggttgcacgttttcaaaaaatacctaatggtgaaaatgagacaatgattcctgtattgacatcaaaaaaagcaagtgaattaccagtcagtgaagttgcaagcattctccaagctgatcttcagaatggtctaaacaaatgtgaagttagtcataggcgagcctttcatggctggaatgagtttgatattagtgaagatgagccactgtggaagaagtatatttctcagtttaaaaatccccttattatgctgcttctggcttctgcagtcatcagtgttttaatgcatcagtttgatgatgccgtcagtatcactgtggcaatacttatcgttgttacagttgcctttgttcaggaatatcgttcagaaaaatctcttgaagaattgagtaaacttgtgccaccagaatgccattgtgtgcgtgaaggaaaattggagcatacacttgcccgagacttggttccaggtgatacagtttgcctttctgttggggatagagttcctgctgacttacgcttgtttgaggctgtggatctttccattgatgagtccagcttgacaggtgagacaacgccttgttctaaggtgacagctcctcagccagctgcaactaatggagatcttgcatcgagaagtaacattgcctttatgggaacactggtcagatgtggcaaagcaaagggtgttgtcattggaacaggagaaaattctgaatttggggaggtttttaaaatgatgcaagcagaagaggcaccaaaaacccctctgcagaagagcatggacctcttaggaaaacaactttccttttactcctttggtataataggaatcatcatgttggttggctggttactgggaaaagatatcctggaaatgtttactattagtgtaagtttggctgtagcagcaattcctgaaggtctccccattgtggtcacagtgacgctagctcttggtgttatgagaatggtgaagaaaagggccattgtgaaaaagctgcctattgttgaaactctgggctgctgtaatgtgatttgttcagataaaactggaacactgacgaagaatgaaatgactgttactcacatatttacttcagatggtctgcatgctgaggttactggagttggctataatcaatttggggaagtgattgttgatggtgatgttgttcatggattctataacccagctgttagcagaattgttgaggcgggctgtgtgtgcaatgatgctgtaattagaaacaatactctaatggggaagccaacagaaggggccttaattgctcttgcaatgaagatgggtcttgatggacttcaacaagactacatcagaaaagctgaatacccttttagctctgagcaaaagtggatggctgttaagtgtgtacaccgaacacagcaggacagaccagagatttgttttatgaaaggtgcttacgaacaagtaattaagtactgtactacataccagagcaaagggcagaccttgacacttactcagcagcagagagatgtgtaccaacaagagaaggcacgcatgggctcagcgggactcagagttcttgctttggcttctggtcctgaactgggacagctgacatttcttggcttggtgggaatcattgatccacctagaactggtgtgaaagaagctgttacaacactcattgcctcaggagtatcaataaaaatgattactggagattcacaggagactgcagttgcaatcgccagtcgtctgggattgtattccaaaacttcccagtcagtctcaggagaagaaatagatgcaatggatgttcagcagctttcacaaatagtaccaaaggttgcagtattttacagagctagcccaaggcacaagatgaaaattattaagtcgctacagaagaacggttcagttgtagccatgacaggagatggagtaaatgatgcagttgctctgaaggctgcagacattggagttgcgatgggccagactggtacagatgtttgcaaagaggcagcagacatgatcctagtggatgatgattttcaaaccataatgtctgcaatcgaagagggtaaagggatttataataacattaaaaatttcgttagattccagctgagcacgagtatagcagcattaactttaatctcattggctacattaatgaactttcctaatcctctcaatgccatgcagattttgtggatcaatattattatggatggacccccagctcagagccttggagtagaaccagtggataaagatgtcattcgtaaacctcctcgcaactggaaagacagcattttgactaaaaacttgatacttaaaatacttgtttcatcaataatcattgtttgtgggactttgtttgtcttctggcgtgagctacgagacaatgtgattacacctcgagacacaacaatgaccttcacatgctttgtgttttttgacatgttcaatgcactaagttccagatcccagaccaagtctgtgtttgagattggactctgcagtaatagaatgttttgctatgcagttcttggatccatcatgggacaattactagttatttactttcctccgcttcagaaggtttttcagactgagagcctaagcatactggatctgttgtttcttttgggtctcacctcatcagtgtgcatagtggcagaaattataaagaaggttgaaaggagcagggaaaagatccagaagcatgttagttcgacatcatcatcttttcttgaagtatgagcggccgcctcgagtctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 2 - DNA, 6715 bp, synthetic construct
  source 1..6715: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctaaggcaattgactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccttcgagctagaaaggaaccaattcagtcgacgctagcaagcttggtaccggatccgaattccccgaaccgacagtcggtctcttcaccaaggccattcgcgccaccatggagcaaaagctcatttctgaggaagatctcaatggtggtggtggtgggttaattaacgtcgagggacgcgtctccgagttcctgaagaaactcggcttctcgggcgggggccgccagtaccaggcgctggagaaggacgaagaggaagccttgattgatgaacagagtgagctgaaagccatcgagaaagagaagaaggtgacagccctgccccccaaggaagcgtgcaaatgccagaaagaggatttggccagagcgttttgtgtggacttacacactgggctgtcggagttctcggtgacgcagcgccggctggcccatggctggaatgagtttgttgctgacaacagcgaacctgtgtggaagaaatacctggatcagtttaagaaccccctgatcctgctgctgctgggctctgccctggtgagtgtcctcaccaaggagtatgaggacgccgtcagcatcgccacggcagtgcttgtcgtggtcactgtcgccttcatccaggagtacaggtcggagaaatctctggaagagctgaccaagatggttcctccagaatgtaactgcctaagagaaggaaaactccagcacctgcttgctcgagaactggttcctggtgatgtcgtatctctctcgatcggagaccggatccctgcagacatccgactcactgaggtcacggacctcttggtggatgaatccagtttcaccggggaagccgagccatgtagtaaaacagacagccccttgacaggcggtggggacctcaccaccctcagcaacatcgtcttcatggggaccctggtgcagtatgggaggggccagggggtcgtgattggaacaggggaaagctctcagttcggagaagtgtttaagatgatgcaggctgaagagacacctaaaactcctttgcagaaaagcatggacaggctaggaaagcaactgacactcttctcctttggcataatcggtctcatcatgctcattggctggtcgcaagggaaacaactcctgagtatgttcacgatcggggtcagcctggctgtggcggctattccagagggtctgcccatcgtcgtcatggtgacgctggtcctgggagtgctgcggatggccaagaagcgggtcatcgtgaagaagttacccatcgtggagactttaggttgctgcagcgttctctgttctgacaagacggggactctgactgccaatgaaatgacagtgacccagcttgtaacgtcagatgggcttcgtgccgaggtcagcggagttgggtatgacggtcaagggactgtgtgtcttctaccatccaaggaagtcattaaggaattttccaatgtctcagtgggaaagttagtggaggcgggctgtgttgccaacaatgcggtcatcagaaagaacgccgtgatggggcagcccaccgagggtgcattgatggccctggcgatgaagatggacttaagtgatattaaaaattcatatataagaaaaaaagagattccattcagttcagagcagaagtggatggcggtgaaatgcagtctgaagactgaggatcaggaagacatttacttcatgaaaggggccttggaagaggtgatccgctactgcaccatgtacaacaacgggggcatccccctgccgctgacgccccagcagaggtcattctgcctgcaggaagagaagaggatggggtcgctcggtttgcgggtgctggccctggcttctgggcccgagctggggcggctgacgtttctaggtcttgtgggcatcattgaccccccgagagttggcgtgaaggaagcagtccaggttctctccgagtctggtgtgtctgtgaagatgataacgggggatgccctggagacggccttggccataggaagaaacatcggcctgtgcaacgggaagctgcaagccatgtccggggaggaggtggacagcgtggagaagggcgagctggccgaccgcgtggggaaggtgtccgtgttcttcaggaccagcccaaagcacaagctcaaaatcatcaaggctctgcaggagtcaggggcgatcgtggccatgactggggatggggtgaacgacgcagtggccctgaagtctgcagacattgggatcgccatggggcagacagggacggacgtcagcaaagaggccgccaacatgatcctggtggatgatgacttctcagccatcatgaatgcagtggaggaaggcaagggtattttttacaacatcaaaaactttgtccgattccagctgagcacgagcatctccgccctgagtctcatcactctgtccaccgtgttcaacctgcccagccccctcaacgccatgcagatcctatggatcaacatcatcatggatgggccaccggcgcagagcttgggggtagagcccgttgacaaagacgccttcaggcagccaccacggagtgtgcgggacaccatcctcagcagagccctcatcctgaagatcctcatgtccgcggccatcatcatcagcgggaccctctttatcttctggaaggagatgcctgaagacagagcaagcactccccgcaccacgacgatgacgttcacttgttttgtgtttttcgatctcttcaacgccttgacctgccgctctcagaccaagctgatatttgagatcggctttctcaggaaccacatgttcctctactccgtcctggggtccatcctggggcagctggcggtcatttacatccccccgctgcagagggtcttccagacggagaacctgggagcgcttgatttgctgtttttaactggattggcctcatccgtcttcattttgtcagagctcctcaaactatgtgaaaaatactgttgcagccccaagagagtccagatgcaccctgaagatgtgtaggcggccgcctcgagtctagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 3 - DNA, 2817 bp, synthetic construct
  source 1..2817: mol_type = other DNA
atggagcaaaagctcatttctgaggaagatctcaatggtggtggtggtgggttaattaagaaggttgcacgttttcaaaaaatacctaatggtgaaaatgagacaatgattcctgtattgacatcaaaaaaagcaagtgaattaccagtcagtgaagttgcaagcattctccaagctgatcttcagaatggtctaaacaaatgtgaagttagtcataggcgagcctttcatggctggaatgagtttgatattagtgaagatgagccactgtggaagaagtatatttctcagtttaaaaatccccttattatgctgcttctggcttctgcagtcatcagtgttttaatgcatcagtttgatgatgccgtcagtatcactgtggcaatacttatcgttgttacagttgcctttgttcaggaatatcgttcagaaaaatctcttgaagaattgagtaaacttgtgccaccagaatgccattgtgtgcgtgaaggaaaattggagcatacacttgcccgagacttggttccaggtgatacagtttgcctttctgttggggatagagttcctgctgacttacgcttgtttgaggctgtggatctttccattgatgagtccagcttgacaggtgagacaacgccttgttctaaggtgacagctcctcagccagctgcaactaatggagatcttgcatcgagaagtaacattgcctttatgggaacactggtcagatgtggcaaagcaaagggtgttgtcattggaacaggagaaaattctgaatttggggaggtttttaaaatgatgcaagcagaagaggcaccaaaaacccctctgcagaagagcatggacctcttaggaaaacaactttccttttactcctttggtataataggaatcatcatgttggttggctggttactgggaaaagatatcctggaaatgtttactattagtgtaagtttggctgtagcagcaattcctgaaggtctccccattgtggtcacagtgacgctagctcttggtgttatgagaatggtgaagaaaagggccattgtgaaaaagctgcctattgttgaaactctgggctgctgtaatgtgatttgttcagataaaactggaacactgacgaagaatgaaatgactgttactcacatatttacttcagatggtctgcatgctgaggttactggagttggctataatcaatttggggaagtgattgttgatggtgatgttgttcatggattctataacccagctgttagcagaattgttgaggcgggctgtgtgtgcaatgatgctgtaattagaaacaatactctaatggggaagccaacagaaggggccttaattgctcttgcaatgaagatgggtcttgatggacttcaacaagactacatcagaaaagctgaatacccttttagctctgagcaaaagtggatggctgttaagtgtgtacaccgaacacagcaggacagaccagagatttgttttatgaaaggtgcttacgaacaagtaattaagtactgtactacataccagagcaaagggcagaccttgacacttactcagcagcagagagatgtgtaccaacaagagaaggcacgcatgggctcagcgggactcagagttcttgctttggcttctggtcctgaactgggacagctgacatttcttggcttggtgggaatcattgatccacctagaactggtgtgaaagaagctgttacaacactcattgcctcaggagtatcaataaaaatgattactggagattcacaggagactgcagttgcaatcgccagtcgtctgggattgtattccaaaacttcccagtcagtctcaggagaagaaatagatgcaatggatgttcagcagctttcacaaatagtaccaaaggttgcagtattttacagagctagcccaaggcacaagatgaaaattattaagtcgctacagaagaacggttcagttgtagccatgacaggagatggagtaaatgatgcagttgctctgaaggctgcagacattggagttgcgatgggccagactggtacagatgtttgcaaagaggcagcagacatgatcctagtggatgatgattttcaaaccataatgtctgcaatcgaagagggtaaagggatttataataacattaaaaatttcgttagattccagctgagcacgagtatagcagcattaactttaatctcattggctacattaatgaactttcctaatcctctcaatgccatgcagattttgtggatcaatattattatggatggacccccagctcagagccttggagtagaaccagtggataaagatgtcattcgtaaacctcctcgcaactggaaagacagcattttgactaaaaacttgatacttaaaatacttgtttcatcaataatcattgtttgtgggactttgtttgtcttctggcgtgagctacgagacaatgtgattacacctcgagacacaacaatgaccttcacatgctttgtgttttttgacatgttcaatgcactaagttccagatcccagaccaagtctgtgtttgagattggactctgcagtaatagaatgttttgctatgcagttcttggatccatcatgggacaattactagttatttactttcctccgcttcagaaggtttttcagactgagagcctaagcatactggatctgttgtttcttttgggtctcacctcatcagtgtgcatagtggcagaaattataaagaaggttgaaaggagcagggaaaagatccagaagcatgttagttcgacatcatcatcttttcttgaagtatga

SEQ ID NO: 4 - DNA, 2898 bp, synthetic construct
  source 1..2898: mol_type = other DNA
atggagcaaaagctcatttctgaggaagatctcaatggtggtggtggtgggttaattaacgtcgagggacgcgtctccgagttcctgaagaaactcggcttctcgggcgggggccgccagtaccaggcgctggagaaggacgaagaggaagccttgattgatgaacagagtgagctgaaagccatcgagaaagagaagaaggtgacagccctgccccccaaggaagcgtgcaaatgccagaaagaggatttggccagagcgttttgtgtggacttacacactgggctgtcggagttctcggtgacgcagcgccggctggcccatggctggaatgagtttgttgctgacaacagcgaacctgtgtggaagaaatacctggatcagtttaagaaccccctgatcctgctgctgctgggctctgccctggtgagtgtcctcaccaaggagtatgaggacgccgtcagcatcgccacggcagtgcttgtcgtggtcactgtcgccttcatccaggagtacaggtcggagaaatctctggaagagctgaccaagatggttcctccagaatgtaactgcctaagagaaggaaaactccagcacctgcttgctcgagaactggttcctggtgatgtcgtatctctctcgatcggagaccggatccctgcagacatccgactcactgaggtcacggacctcttggtggatgaatccagtttcaccggggaagccgagccatgtagtaaaacagacagccccttgacaggcggtggggacctcaccaccctcagcaacatcgtcttcatggggaccctggtgcagtatgggaggggccagggggtcgtgattggaacaggggaaagctctcagttcggagaagtgtttaagatgatgcaggctgaagagacacctaaaactcctttgcagaaaagcatggacaggctaggaaagcaactgacactcttctcctttggcataatcggtctcatcatgctcattggctggtcgcaagggaaacaactcctgagtatgttcacgatcggggtcagcctggctgtggcggctattccagagggtctgcccatcgtcgtcatggtgacgctggtcctgggagtgctgcggatggccaagaagcgggtcatcgtgaagaagttacccatcgtggagactttaggttgctgcagcgttctctgttctgacaagacggggactctgactgccaatgaaatgacagtgacccagcttgtaacgtcagatgggcttcgtgccgaggtcagcggagttgggtatgacggtcaagggactgtgtgtcttctaccatccaaggaagtcattaaggaattttccaatgtctcagtgggaaagttagtggaggcgggctgtgttgccaacaatgcggtcatcagaaagaacgccgtgatggggcagcccaccgagggtgcattgatggccctggcgatgaagatggacttaagtgatattaaaaattcatatataagaaaaaaagagattccattcagttcagagcagaagtggatggcggtgaaatgcagtctgaagactgaggatcaggaagacatttacttcatgaaaggggccttggaagaggtgatccgctactgcaccatgtacaacaacgggggcatccccctgccgctgacgccccagcagaggtcattctgcctgcaggaagagaagaggatggggtcgctcggtttgcgggtgctggccctggcttctgggcccgagctggggcggctgacgtttctaggtcttgtgggcatcattgaccccccgagagttggcgtgaaggaagcagtccaggttctctccgagtctggtgtgtctgtgaagatgataacgggggatgccctggagacggccttggccataggaagaaacatcggcctgtgcaacgggaagctgcaagccatgtccggggaggaggtggacagcgtggagaagggcgagctggccgaccgcgtggggaaggtgtccgtgttcttcaggaccagcccaaagcacaagctcaaaatcatcaaggctctgcaggagtcaggggcgatcgtggccatgactggggatggggtgaacgacgcagtggccctgaagtctgcagacattgggatcgccatggggcagacagggacggacgtcagcaaagaggccgccaacatgatcctggtggatgatgacttctcagccatcatgaatgcagtggaggaaggcaagggtattttttacaacatcaaaaactttgtccgattccagctgagcacgagcatctccgccctgagtctcatcactctgtccaccgtgttcaacctgcccagccccctcaacgccatgcagatcctatggatcaacatcatcatggatgggccaccggcgcagagcttgggggtagagcccgttgacaaagacgccttcaggcagccaccacggagtgtgcgggacaccatcctcagcagagccctcatcctgaagatcctcatgtccgcggccatcatcatcagcgggaccctctttatcttctggaaggagatgcctgaagacagagcaagcactccccgcaccacgacgatgacgttcacttgttttgtgtttttcgatctcttcaacgccttgacctgccgctctcagaccaagctgatatttgagatcggctttctcaggaaccacatgttcctctactccgtcctggggtccatcctggggcagctggcggtcatttacatccccccgctgcagagggtcttccagacggagaacctgggagcgcttgatttgctgtttttaactggattggcctcatccgtcttcattttgtcagagctcctcaaactatgtgaaaaatactgttgcagccccaagagagtccagatgcaccctgaagatgtgtag

SEQ ID NO: 5 - DNA, 30 bp, synthetic construct
  source 1..30: mol_type = other DNA
gagcaaaagctcatttctgaggaagatctc

SEQ ID NO: 6 - DNA, 27 bp, synthetic construct
  source 1..27: mol_type = other DNA
aatggtggtggtggtgggttaattaag

SEQ ID NO: 7 - DNA, 2754 bp, Homo sapiens
  source 1..2754: mol_type = genomic DNA
aaggttgcacgttttcaaaaaatacctaatggtgaaaatgagacaatgattcctgtattgacatcaaaaaaagcaagtgaattaccagtcagtgaagttgcaagcattctccaagctgatcttcagaatggtctaaacaaatgtgaagttagtcataggcgagcctttcatggctggaatgagtttgatattagtgaagatgagccactgtggaagaagtatatttctcagtttaaaaatccccttattatgctgcttctggcttctgcagtcatcagtgttttaatgcatcagtttgatgatgccgtcagtatcactgtggcaatacttatcgttgttacagttgcctttgttcaggaatatcgttcagaaaaatctcttgaagaattgagtaaacttgtgccaccagaatgccattgtgtgcgtgaaggaaaattggagcatacacttgcccgagacttggttccaggtgatacagtttgcctttctgttggggatagagttcctgctgacttacgcttgtttgaggctgtggatctttccattgatgagtccagcttgacaggtgagacaacgccttgttctaaggtgacagctcctcagccagctgcaactaatggagatcttgcatcgagaagtaacattgcctttatgggaacactggtcagatgtggcaaagcaaagggtgttgtcattggaacaggagaaaattctgaatttggggaggtttttaaaatgatgcaagcagaagaggcaccaaaaacccctctgcagaagagcatggacctcttaggaaaacaactttccttttactcctttggtataataggaatcatcatgttggttggctggttactgggaaaagatatcctggaaatgtttactattagtgtaagtttggctgtagcagcaattcctgaaggtctccccattgtggtcacagtgacgctagctcttggtgttatgagaatggtgaagaaaagggccattgtgaaaaagctgcctattgttgaaactctgggctgctgtaatgtgatttgttcagataaaactggaacactgacgaagaatgaaatgactgttactcacatatttacttcagatggtctgcatgctgaggttactggagttggctataatcaatttggggaagtgattgttgatggtgatgttgttcatggattctataacccagctgttagcagaattgttgaggcgggctgtgtgtgcaatgatgctgtaattagaaacaatactctaatggggaagccaacagaaggggccttaattgctcttgcaatgaagatgggtcttgatggacttcaacaagactacatcagaaaagctgaatacccttttagctctgagcaaaagtggatggctgttaagtgtgtacaccgaacacagcaggacagaccagagatttgttttatgaaaggtgcttacgaacaagtaattaagtactgtactacataccagagcaaagggcagaccttgacacttactcagcagcagagagatgtgtaccaacaagagaaggcacgcatgggctcagcgggactcagagttcttgctttggcttctggtcctgaactgggacagctgacatttcttggcttggtgggaatcattgatccacctagaactggtgtgaaagaagctgttacaacactcattgcctcaggagtatcaataaaaatgattactggagattcacaggagactgcagttgcaatcgccagtcgtctgggattgtattccaaaacttcccagtcagtctcaggagaagaaatagatgcaatggatgttcagcagctttcacaaatagtaccaaaggttgcagtattttacagagctagcccaaggcacaagatgaaaattattaagtcgctacagaagaacggttcagttgtagccatgacaggagatggagtaaatgatgcagttgctctgaaggctgcagacattggagttgcgatgggccagactggtacagatgtttgcaaagaggcagcagacatgatcctagtggatgatgattttcaaaccataatgtctgcaatcgaagagggtaaagggatttataataacattaaaaatttcgttagattccagctgagcacgagtatagcagcattaactttaatctcattggctacattaatgaactttcctaatcctctcaatgccatgcagattttgtggatcaatattattatggatggacccccagctcagagccttggagtagaaccagtggataaagatgtcattcgtaaacctcctcgcaactggaaagacagcattttgactaaaaacttgatacttaaaatacttgtttcatcaataatcattgtttgtgggactttgtttgtcttctggcgtgagctacgagacaatgtgattacacctcgagacacaacaatgaccttcacatgctttgtgttttttgacatgttcaatgcactaagttccagatcccagaccaagtctgtgtttgagattggactctgcagtaatagaatgttttgctatgcagttcttggatccatcatgggacaattactagttatttactttcctccgcttcagaaggtttttcagactgagagcctaagcatactggatctgttgtttcttttgggtctcacctcatcagtgtgcatagtggcagaaattataaagaaggttgaaaggagcagggaaaagatccagaagcatgttagttcgacatcatcatcttttcttgaagta

SEQ ID NO: 8 - DNA, 2835 bp, Homo sapiens
  source 1..2835: mol_type = genomic DNA
gtcgagggacgcgtctccgagttcctgaagaaactcggcttctcgggcgggggccgccagtaccaggcgctggagaaggacgaagaggaagccttgattgatgaacagagtgagctgaaagccatcgagaaagagaagaaggtgacagccctgccccccaaggaagcgtgcaaatgccagaaagaggatttggccagagcgttttgtgtggacttacacactgggctgtcggagttctcggtgacgcagcgccggctggcccatggctggaatgagtttgttgctgacaacagcgaacctgtgtggaagaaatacctggatcagtttaagaaccccctgatcctgctgctgctgggctctgccctggtgagtgtcctcaccaaggagtatgaggacgccgtcagcatcgccacggcagtgcttgtcgtggtcactgtcgccttcatccaggagtacaggtcggagaaatctctggaagagctgaccaagatggttcctccagaatgtaactgcctaagagaaggaaaactccagcacctgcttgctcgagaactggttcctggtgatgtcgtatctctctcgatcggagaccggatccctgcagacatccgactcactgaggtcacggacctcttggtggatgaatccagtttcaccggggaagccgagccatgtagtaaaacagacagccccttgacaggcggtggggacctcaccaccctcagcaacatcgtcttcatggggaccctggtgcagtatgggaggggccagggggtcgtgattggaacaggggaaagctctcagttcggagaagtgtttaagatgatgcaggctgaagagacacctaaaactcctttgcagaaaagcatggacaggctaggaaagcaactgacactcttctcctttggcataatcggtctcatcatgctcattggctggtcgcaagggaaacaactcctgagtatgttcacgatcggggtcagcctggctgtggcggctattccagagggtctgcccatcgtcgtcatggtgacgctggtcctgggagtgctgcggatggccaagaagcgggtcatcgtgaagaagttacccatcgtggagactttaggttgctgcagcgttctctgttctgacaagacggggactctgactgccaatgaaatgacagtgacccagcttgtaacgtcagatgggcttcgtgccgaggtcagcggagttgggtatgacggtcaagggactgtgtgtcttctaccatccaaggaagtcattaaggaattttccaatgtctcagtgggaaagttagtggaggcgggctgtgttgccaacaatgcggtcatcagaaagaacgccgtgatggggcagcccaccgagggtgcattgatggccctggcgatgaagatggacttaagtgatattaaaaattcatatataagaaaaaaagagattccattcagttcagagcagaagtggatggcggtgaaatgcagtctgaagactgaggatcaggaagacatttacttcatgaaaggggccttggaagaggtgatccgctactgcaccatgtacaacaacgggggcatccccctgccgctgacgccccagcagaggtcattctgcctgcaggaagagaagaggatggggtcgctcggtttgcgggtgctggccctggcttctgggcccgagctggggcggctgacgtttctaggtcttgtgggcatcattgaccccccgagagttggcgtgaaggaagcagtccaggttctctccgagtctggtgtgtctgtgaagatgataacgggggatgccctggagacggccttggccataggaagaaacatcggcctgtgcaacgggaagctgcaagccatgtccggggaggaggtggacagcgtggagaagggcgagctggccgaccgcgtggggaaggtgtccgtgttcttcaggaccagcccaaagcacaagctcaaaatcatcaaggctctgcaggagtcaggggcgatcgtggccatgactggggatggggtgaacgacgcagtggccctgaagtctgcagacattgggatcgccatggggcagacagggacggacgtcagcaaagaggccgccaacatgatcctggtggatgatgacttctcagccatcatgaatgcagtggaggaaggcaagggtattttttacaacatcaaaaactttgtccgattccagctgagcacgagcatctccgccctgagtctcatcactctgtccaccgtgttcaacctgcccagccccctcaacgccatgcagatcctatggatcaacatcatcatggatgggccaccggcgcagagcttgggggtagagcccgttgacaaagacgccttcaggcagccaccacggagtgtgcgggacaccatcctcagcagagccctcatcctgaagatcctcatgtccgcggccatcatcatcagcgggaccctctttatcttctggaaggagatgcctgaagacagagcaagcactccccgcaccacgacgatgacgttcacttgttttgtgtttttcgatctcttcaacgccttgacctgccgctctcagaccaagctgatatttgagatcggctttctcaggaaccacatgttcctctactccgtcctggggtccatcctggggcagctggcggtcatttacatccccccgctgcagagggtcttccagacggagaacctgggagcgcttgatttgctgtttttaactggattggcctcatccgtcttcattttgtcagagctcctcaaactatgtgaaaaatactgttgcagccccaagagagtccagatgcaccctgaagatgtg